(19) United States
(12) Patent Application Publication
(10) Pub. No.: US 2019/0210010 A1
(43) Pub. Date: Jul. 11, 2019

(54) METHOD FOR THE HYDROGENATION OF ORGANIC COMPOUNDS IN THE PRESENCE OF CO AND A FIXED CATALYST BED WHICH CONTAINS MONOLITHIC SHAPED CATALYST BODY

(71) Applicant: BASF SE, Ludwigshafen am Rhein (DE)

(72) Inventors: Rolf Pinkos, Ludwigshafen (DE); Irene de Wispelaere, Antwerpen (NL); Michael Schwarz, Ludwigshafen (DE); Michael Schreiber, Ludwigshafen (DE); Zeljko Kotanjac, Ludwigshafen (DE); Michael Nilles, Ludwigshafen (DE)

(21) Appl. No.: 16/335,785

(22) PCT Filed: Sep. 14, 2017
(86) PCT No.: PCT/EP2017/073145

§ 371 Date: Mar. 22, 2019

(30) Foreign Application Priority Data
Sep. 23, 2016 (EP) 16190428.9

(51) Int. Cl. B01J 25/02; B01J 35/04; B01J 23/72; B01J 35/00; B01J 35/10; B01J 35/06; B01J 37/18; B01J 37/08; B01J 37/02; B01J 37/00; C07C 29/145; C07C 29/17; C07C 29/141

(52) U.S. Cl. CPC B01J 25/02; B01J 35/04; B01J 23/72; B01J 35/0026; B01J 35/10; B01J 35/06; C07C 29/141; B01J 37/08; B01J 37/0215; B01J 37/0018; C07C 29/145; C07C 29/172; B01J 37/18

(57) ABSTRACT

A process for hydrogenating a hydrogenatable organic compound in a reactor including a fixed catalyst bed. The fixed catalyst bed includes monolithic shaped catalyst bodies having pores and/or channels. The catalyst bodies include at least one element selected from Ni, Fe, Co, Cu, Cr, Pt, Ag, Au, Pd, Mn, Re, Ru, Rh and Ir. The CO content in the gas phase within the reactor during hydrogenation is within a range from 0.1 to 10,000 ppm by volume. In any section in the normal plane to flow direction through the fixed catalyst bed, at least 90% of the pores and channels have an area of not more than 3 mm².

### BACKGROUND OF THE INVENTION

The present invention relates to a process for hydrogenating organic compounds in the presence of CO and of a fixed catalyst bed comprising immobilized structured shaped catalyst bodies.

### PRIOR ART

It is known in principle that hydrogenation reactions can be conducted in the presence of carbon monoxide (CO). The CO may firstly be added to the hydrogen used for hydrogenation and/or originate from the feedstocks or the intermediates, by-products or products thereof. If catalysts comprising active components sensitive to CO are used for hydrogenation, a known countermeasure is that of conducting the hydrogenation at a high hydrogen pressure and/or a low catalyst hourly space velocity. Otherwise, the conversion can be incomplete, such that, for example, a postreaction in at least one further reactor is absolutely necessary. There can likewise be increased formation of by-products. Drawbacks associated with the use of high hydrogen pressures are the formation of methane resulting from hydrogenation of CO and hence elevated consumption of hydrogen and elevated capital costs.

U.S. Pat. No. 6,262,317 (DE 196 41 707 A1) describes the hydrogenation of butyne-1,4-diol with hydrogen in the liquid continuous phase in the presence of a heterogeneous hydrogenation catalyst at temperatures of 20 to 300° C., a pressure of 1 to 200 bar and values of the liquid-side volume-based mass transfer coefficient kLa of 0.1 s⁻¹ to 1 s⁻¹. The reaction can be effected either in the presence of a catalyst suspended in the reaction medium or in a fixed bed reactor operated in cocurrent in cycle gas mode. It is stated in quite general terms that it is possible to provide fixed bed reactors by directly coating structure packings as typically used in bubble columns with catalytically active substances. However, no further details of this are given. In the working examples, suspension catalysts or reactor packings based on Raschig rings having a diameter of 5 mm were used.

For hydrogenation in fixed bed mode, a ratio of gas stream supplied to gas stream leaving the reactor of 0.99:1 to 0.4:1 is described, meaning that at least 60% of the gas supplied is still present at the end of the reactor.

In suspension mode, good hydrogenation results are described in example 1 with a space velocity of about 0.4 kg of butynediol/liter of reaction space x h. If the space velocity is increased to 0.7 (example 2), there is a decline in the butanediol yield and a rise in the proportion of unwanted by-products, such as 2-methylbutanediol, butanol and propanol. A problem with the hydrogenations in suspension is the handling of the suspended catalyst, which has to remain in the reactor, and so a filter system is absolutely necessary for retention of the catalyst. Filters of this kind have a tendency to become blocked with catalyst particles, and so they either have to be cleaned periodically in a costly and inconvenient manner or the running time is correspondingly short before passage through the filter becomes uneconomic. In examples 5 and 6, using supported catalysts too, a filter was still used in order to keep the particles of the catalyst bed in the reactor. The space velocity corresponded to about 0.25 kg of butynediol/liter of reaction space x h. The total amount of the 2-methylbutanediol, butanol and propanol by-products is relatively high at 6%. The implementation of the process described in DE 196 41 707 A1 is technically complex for the reasons mentioned. Moreover, it is necessary in the case of fixed bed mode to provide a gaseous circulation stream, since at least 60% of the gas supply to the reactor exits again at the end of the reactor. In the case of such a cycle gas mode, however, the risk of accumulation of unwanted components in the gas stream is particularly high; this is particularly true of CO.

DE 199 629 07 A1 describes a process for preparing C₁₀-C₃₀-alkenes by partial hydrogenation of alkynes over fixed bed supported catalysts, wherein CO is added to the hydrogenation gas. The hydrogenation-active metal used is exclusively palladium. Suitable starting materials specifically mentioned are dehydrolinalool, hydrodehydrolinalool, 1-ethynyl-2,6,6-trimethylcyclohexanol, 17-ethynylandrost-5-ene-3β,17β-diol, 3,7,11,15-tetrannethyl-1-hexadecyn-3-ol (dehydroisophytol), 3,7,11-trinnethyl-6-dodecen-1-yn-3-ol (dehydrodihydronerolidol), 4-methyl-4-hydroxy-2-decyne, 1,1-diethoxy-2-octyne and bis(tetrahydro-2-pyranyloxy)-2-butyne.

EP 0 754 664 A2 describes a process for preparing alkenes by partial hydrogenation of alkynes over fixed bed supported catalysts, wherein CO is added to the hydrogenation gas. The hydrogenation-active metal used is again exclusively palladium. A suitable reactant mentioned alongside a great multitude of others is butyne-1,4-diol. However, the working examples describe only the selective hydrogenation of 2-dehydrolinalool to 2-linalool.

DE 433 32 93 A1 describes partial hydrogenation of butyne-1,4-diol to butene-1,4-diol at 60° C. over a structured Pd catalyst. There is no mention of CO formation or the contents thereof. Nor anything about the amount of hydrogen which was utilized, but only of the pressure (15 bar). It can thus be assumed that the hydrogenation was not conducted continuously; instead, the reactant was merely pumped in circulation in trickle mode without significant hydrogen flow.

Known types of catalysts for hydrogenation reactions are precipitation catalysts, supported catalysts or Raney metal catalysts. Raney metal catalysts have found broad commercial use, specifically for hydrogenation of mono- or polyunsaturated organic compounds. Typically, Raney catalysts are alloys comprising at least one catalytically active metal and at least one alloy component soluble (leachable) in alkalis. Typical catalytically active metals are, for example, Ni, Fe, Co, Cu, Cr, Pt, Ag, Au and Pd, and typical leachable alloy components are, for example, Al, Zn and Si. Raney metal catalysts of this kind and processes for preparation thereof are described, for example, in U.S. Pat. Nos. 1,628,190, 1,915,473 and 1,563,587. Before they are used in heterogeneously catalyzed chemical reactions, specifically in a hydrogenation reaction, Raney metal alloys generally have to be subjected to an activation.

Standard processes for activating Raney metal catalysts comprise the grinding of the alloy to give a fine powder if it is not already in powder form as produced. For activation, the powder is subjected to a treatment with an aqueous alkali, with partial removal of the leachable metal from the alloy, leaving the highly active non-leachable metal. The powders thus activated are pyrophoric and are typically stored under water or organic solvents, in order to avoid contact with oxygen and associated deactivation of the Raney metal catalysts.

In a known process for activation of suspended Raney nickel catalysts, a nickel-aluminum alloy is treated with 15% to 20% by weight sodium hydroxide solution at temperatures of 100° C. or higher. U.S. Pat. No. 2,948,687 describes preparing a Raney nickel-molybdenum catalyst from a ground Ni—Mo—Al alloy having particle sizes in the region of 80 mesh (about 0.177 mm) or finer, by first treating the alloy at 50° C. with 20% by weight NaOH solution and raising the temperature to 100 to 115° C.

A crucial disadvantage of pulverulent Raney metal catalysts is the need to separate them from the reaction medium of the catalyzed reaction by costly sedimentation and/or filtration methods.

It is known that Raney metal catalysts can also be used in the form of coarser particles. For instance, U.S. Pat. No. 3,448,060 describes the preparation of structured Raney metal catalysts, wherein, in a first embodiment, an inert support material is coated with an aqueous suspension of a pulverulent nickel-aluminum alloy and freshly precipitated aluminium hydroxide. The structure thus obtained is dried, heated and contacted with water, releasing hydrogen. Subsequently, the structure is hardened. Leaching with an alkali metal hydroxide solution is envisaged as an option. In a second embodiment, an aqueous suspension of a pulverulent nickel-aluminum alloy and freshly precipitated aluminium hydroxide is subjected to shaping without use of a support material. The structure thus obtained is activated analogously to the first embodiment.

Further Raney metal catalysts suitable for use in fixed bed catalysts may include hollow bodies or spheres or have some other kind of support. Catalysts of this kind are described, for example, in EP 0 842 699, EP 1 068 900, U.S. Pat. Nos. 6,747,180, 2,895,819 and US 2009/0018366.

U.S. Pat. No. 2,950,260 describes a process for activating a catalyst composed of a granular nickel-aluminum alloy by treatment with an aqueous alkali solution. Typical particle sizes of this granular alloy are within a range of 1 to 14 mesh (about 20 to 1.4 mm). It has been found that the contacting of a Raney metal alloy, such as an Ni—Al alloy, with an aqueous alkali leads to an exothermic reaction with formation of relatively large amounts of hydrogen. The following reaction equations are intended to elucidate, by way of example, possible reactions which take place when an Ni—Al alloy is contacted with an aqueous alkali such as NaOH:

2NaOH+2Al+2H₂O→2NaAlO₂+3H₂

2Al+6H₂O→2Al(OH)₃+3H₂

2Al(OH)₃→Al₂O₃+3H₂O

The problem addressed by U.S. Pat. No. 2,950,260 is that of providing an activated granular hydrogenation catalyst composed of an Ni—Al alloy with improved activity and service life. For this purpose, the activation is conducted with a 0.5% to 5% by weight NaOH or KOH, the temperature being kept below 35° C. by cooling and contact time being chosen such that not more than 1.5 molar parts of H₂ are released per molar equivalent of alkali. By contrast with a pulverulent suspended catalyst, a distinctly smaller proportion of aluminum is leached out of the structure in the case of treatment of granular Raney metal catalysts. This proportion is within a range of only 5% to 30% by weight, based on the amount of aluminum originally present. Catalyst particles having a porous activated nickel surface and an unchanged metal core are obtained. A disadvantage of the catalysts thus obtained, where only the outermost layer of the particles is catalytically active, is their sensitivity to mechanical stress or abrasion, which can lead to rapid deactivation of the catalyst. The teaching of U.S. Pat. No. 2,950,260 is restricted to granular shaped catalyst bodies, which differ fundamentally from larger structured shaped bodies. Moreover, this document also does not teach that the catalysts may additionally also comprise promoter elements in addition to nickel and aluminum.

It is known that hydrogenation catalysts, such as Raney metal catalysts, can be subjected to doping with at least one promoter element, in order thus to achieve, for example, an improvement in the field, selectivity and/or activity in the hydrogenation. In this way, it is generally possible to obtain products having improved quality. Dopings of this kind are described in U.S. Pat. Nos. 2,953,604, 2,953,605, 2,967,893, 2,950,326, 4,885,410 and 4,153,578.

The use of promoter elements serves, for example, to avoid unwanted side reactions, for example isomerization reactions. Promoter elements are additionally suitable for modifying the activity of the hydrogenation catalysts, in order to achieve, for example, in the case of hydrogenation of reactants having a plurality of hydrogenatable groups, either specific partial hydrogenation of a particular group or two or more particular groups or else full hydrogenation of all hydrogenatable groups. For example, it is known that it is possible to use, for partial hydrogenation of butyne-1,4-diol to butene-1,4-diol, a copper-modified nickel or palladium catalyst (see, for example, GB832141). In principle, the activity and/or selectivity of a catalyst can thus be increased or lowered by doping with at least one promoter metal. Such doping should as far as possible not adversely affect the other hydrogenation properties of the doped catalyst.

For modification of shaped catalyst bodies by doping, the following four methods are known in principle:
the promoter elements are already present in the alloy for preparation of the shaped catalyst bodies (method 1),
the shaped catalyst bodies are contacted with a dopant during the activation (method 2),
the shaped catalyst bodies are contacted with a dopant after the activation (method 3),
the shaped catalyst bodies are contacted with a dopant in the hydrogenation feed stream during the hydrogenation, or a dopant is introduced into the reactor during the hydrogenation in some other way (method 4).

The abovementioned method 1, in which at least one promoter is already present in the alloy for preparation of the shaped catalyst bodies, is described, for example, in U.S. Pat. No. 2,948,687 which has already been mentioned at the outset. According to this, to prepare the catalyst, a finely ground nickel-aluminum-molybdenum alloy is used in order to prepare a molybdenum-containing Raney nickel catalyst.

The abovementioned methods 2 and 3 are described, for example, in US 2010/0174116 A1 (=U.S. Pat. No. 8,889,911). According to this, a doped catalyst is prepared from an Ni/AI alloy, which is modified with at least one promoter metal during and/or after the activation thereof. In this case, the catalyst may optionally already have been subjected to a first doping prior to the activation. The promoter element used for doping by absorption on the surface of the catalyst during and/or after the activation is selected from Mg, Ca, Ba, Ti, Zr, Ce, Nb, Cr, Mo, W, Mn, Re, Fe, Co, Ir, Ni, Cu, Ag, Au, Bi, Rh and Ru. If the catalyst precursor has already been subjected to doping prior to the activation, the promoter element is selected from Ti, Ce, V, Cr, Mo, W, Mn, Re, Fe, Ru, Co, Rh, Ir, Pd, Pt and Bi.

The abovementioned method 3 is also described in GB 2104794. This document relates to Raney nickel catalysts for the reduction of organic compounds, specifically the reduction of carbonyl compounds and the preparation of butane-1,4-diol from butyne-1,4-diol. For preparation of these catalysts, a Raney nickel catalyst is subjected to doping with a molybdenum compound, which may be in solid form or in the form of a dispersion or solution. Other promoter elements, such as Cu, Cr, Co, W, Zr, Pt or Pd, may additionally be used. In a specific embodiment, an already activated commercially available undoped Raney nickel catalyst is suspended in water together with ammonium molybdate and the suspension is stirred until a sufficient amount of molybdenum has been absorbed. In this document, exclusively particulate Raney nickel catalysts are used for doping; specifically, there is no description of the use of structured shaped bodies. There is also no pointer as to how the catalysts can be introduced into a reactor in the form of a structured fixed catalyst bed and as to how the fixed catalyst bed introduced into the reactor can then be activated and doped.

The abovementioned method 4 is described, for example, in U.S. Pat. Nos. 2,967,893 or 2,950,326. According to this, copper is added in the form of copper salts to a nickel catalyst for the hydrogenation of butyne-1,4-diol under aqueous conditions.

According to EP 2 486 976 A1, supported activated Raney metal catalysts are subsequently doped with an aqueous metal salt solution. Specifically, the supports used are the bulk materials customary for the purpose, for example SiO₂-coated glass bodies having a diameter of about 3 mm. There is no description of conducting the doping and optionally even the activation beforehand over a fixed catalyst bed composed of structured shaped catalyst bodies present at a fixed location in a reactor. Thus, it is impossible by the process described in this document to provide a fixed catalyst bed having a gradient with respect to the concentration of the promoter elements in flow direction of the reaction medium of the reaction to be catalyzed.

EP 2 764 916 A1 describes a process for producing shaped foam catalyst bodies suitable for hydrogenations by:
a) providing a shaped metal foam body comprising at least one first metal selected, for example, from Ni, Fe, Co, Cu, Cr, Pt, Ag, Au and Pd,
b) applying at least one second leachable component or a component convertible to a leachable component by alloying, selected, for example, from Al, Zn and Si, to the surface of the shaped metal foam body, and
c) forming an alloy by alloying the shaped metal foam body obtained in step b) at least over part of its surface, and
d) subjecting the alloy obtained in the form of a foam in step c) to a treatment with an agent capable of leaching out the leachable component of the alloy.

This document teaches using 1 to 10 molar, i.e. 4% to 40% by weight, aqueous NaOH for step d). The temperature in step d) is 20 to 98° C., and the treatment time is 1 to 15 minutes. It is mentioned in quite general terms that the shaped foam bodies of the invention can also be formed in situ in a chemical reactor, but without any specific details. EP 2 764 916 A1 also teaches that it is possible to use promoter elements in the production of shaped foam catalyst bodies. The doping can be effected together with the application of the leachable component to the surface of the shaped metal foam body prepared beforehand. The doping can also be effected in a separate step after the activation.

EP 2 764 916 A1 does not contain the slightest details as to the dimensions of the chemical reactor for the use of the shaped foam bodies, the type, amount and dimensions of the shaped bodies introduced into the reactor, and the introduction of the shaped bodies into the reactor. More particularly, there is a lack of any detail as to how a real fixed catalyst bed present in a chemical reactor can first be activated and then doped.

It is an object of the present invention to provide an improved process for hydrogenating organic compounds, which overcomes as many as possible of the aforementioned disadvantages.

It has been found that unsaturated organic compounds can advantageously be hydrogenated to saturated compounds when hydrogenation is effected using monolithic fixed bed catalysts and the CO content in the gas phase within the reactor is within a range from 0.1 to 10 000 ppm by weight, where the conversion is at least 90% and wherein the fixed catalyst bed comprises shaped catalyst bodies having pores and/or channels, and wherein, in any section in the normal plane to flow direction through the fixed catalyst bed, at least 90% of the pores and channels, more preferably at least 98% of the pores and channels, have an area of not more than 3 mm².

### SUMMARY OF THE INVENTION

The invention provides a process for hydrogenating a hydrogenatable organic compound in at least one reactor comprising a fixed catalyst bed comprising monolithic shaped catalyst bodies or consisting of monolithic shaped catalyst bodies comprising at least element selected from Ni, Fe, Co, Cu, Cr, Pt, Ag, Au, Pd, Mn, Re, Ru, Rh and Ir, wherein the CO content in the gas phase within the reactor during the hydrogenation is within a range from 0.1 to 10 000 ppm by volume and wherein the fixed catalyst bed comprises shaped catalyst bodies having pores and/or channels, and wherein, in any section in the normal plane to flow direction through the fixed catalyst bed, at least 90% of the pores and channels, more preferably at least 98% of the pores and channels, have an area of not more than 3 mm².

### PREFERRED EMBODIMENTS OF THE INVENTION

The invention encompasses the following preferred embodiments:
1. A process for hydrogenating a hydrogenatable organic compound in at least one reactor comprising a fixed catalyst bed comprising monolithic shaped catalyst bodies or consisting of monolithic shaped catalyst bodies comprising at least one element selected from Ni, Fe, Co, Cu, Cr, Pt, Ag, Au, Pd, Mn, Re, Ru, Rh and Ir, wherein the CO content in the gas phase within the reactor during the hydrogenation is within a range from 0.1 to 10 000 ppm by volume and wherein the fixed catalyst bed comprises shaped catalyst bodies having pores and/or channels, and wherein, in any section in the normal plane to flow direction through the fixed catalyst bed, at least 90% of the pores and channels, more preferably at least 98% of the pores and channels, have an area of not more than 3 mm².
2. The process according to embodiment 1, wherein the hydrogenatable organic compound is selected from compounds having at least one carbon-carbon double bond, carbon-nitrogen double bond, carbon-oxygen double bond, carbon-carbon triple bond, carbon-nitrogen triple bond or nitrogen-oxygen double bond.
3. The process according to any of the preceding embodiments, wherein the compound used for hydrogenation is selected from butyne-1,4-diol, butene-1,4-diol, 4-hydroxybutyraldehyde, hydroxypivalic acid, hydroxypivalaldehyde, n-butyraldehyde, isobutyraldehyde, n-valeraldehyde, isovaleraldehyde, 2-ethylhex-2-enal, 2-ethylhexanal, the isomeric nonanals, cyclododeca-1,5,9-triene, benzene, furan, furfural, phthalic esters, acetophenone and alkyl-substituted acetophenones.
4. The process according to any of the preceding embodiments, wherein the compound used for hydrogenation is selected from butyne-1,4-diol, butene-1,4-diol, n- and isobutyraldehyde, hydroxypivalaldehyde, 2-ethylhex-2-enal, the isomeric nonanals and 4-isobutylacetophenone.
5. The process according to any of the preceding embodiments, wherein the hydrogenation is conducted continuously.
6. The process according to any of the preceding embodiments, wherein the reactor has an internal volume in the range from 0.1 to 100 m³, preferably from 0.5 to 80 m³.
7. The process according to any of the preceding embodiments, wherein the conversion in the hydrogenation is at least 90 mol %, preferably at least 95 mol %, particularly at least 99 mol %, especially at least 99.5 mol %, based on the total molar amount of hydrogenatable components in the starting material used for hydrogenation.
8. The process according to any of the preceding embodiments, wherein, during the hydrogenation, the CO content in the gas phase within the reactor is within a range from 0.15 to 5000 ppm by volume, especially within a range from 0.2 to 1000 ppm by volume.
9. The process according to any of the preceding embodiments, wherein the reactor has a gradient with respect to the CO concentration in flow direction of the reaction medium through the fixed catalyst bed.
10. The process according to any of the preceding embodiments, wherein the CO content on exit of the reaction medium from the fixed catalyst bed is at least 5 mol %, preferably at least 25 mol % and especially at least 75 mol % higher than the CO content on entry of the reaction medium into the catalytically active fixed bed.
11. The process according to any of the preceding embodiments, wherein the fixed catalyst bed has, in any section in the normal plane to flow direction through the fixed catalyst bed, based on the total area of the section, not more than 5%, preferably not more than 1% and especially not more than 0.1% free area that is not part of the shaped catalyst bodies.
12. The process according to any of the preceding embodiments, wherein the fixed catalyst bed is filled with shaped catalyst bodies to an extent of at least 95% of the reactor cross section over at least 90% length in flow direction through the fixed catalyst bed, preferably to an extent of at least 98% of the reactor cross section, especially at least 99% of the reactor cross section.
13. The process according to any of the preceding embodiments, wherein the flow rate of the liquid reaction mixture through the reactor comprising the fixed catalyst bed is at least 30 m/h, preferably at least 50 m/h, especially at least 80 m/h.
14. The process according to any of the preceding embodiments, wherein the flow rate of the liquid reaction mixture through the reactor comprising the fixed catalyst bed is at most 1000 m/h, preferably at most 500 m/h, especially at most 400 m/h.
15. The process according to any of the preceding embodiments, wherein the reaction mixture of the hydrogenation is at least partly conducted in a liquid circulation stream.
16. The process according to any of the preceding embodiments, wherein the ratio of reaction mixture conducted in the circulation stream to freshly supplied reactant stream is within a range from 1:1 to 1000:1, preferably from 2:1 to 500:1, especially from 5:1 to 200:1.
17. The process according to any of the preceding embodiments, wherein an output is withdrawn from the reactor and subjected to a gas/liquid separation to obtain a hydrogen-containing gas phase and a product-containing liquid phase.
18. The process according to any of the preceding embodiments, wherein the absolute pressure in the hydrogenation is preferably within a range from 1 to 330 bar, more preferably within a range from 5 to 100 bar, especially within a range from 10 to 60 bar.
19. The process according to any of the preceding embodiments, wherein the temperature in the hydrogenation is preferably within a range from 40 to 300° C., more preferably from 70 to 220° C., especially from 80 to 200° C.
20. The process according to any of the preceding embodiments, wherein the fixed catalyst bed has a temperature gradient during the hydrogenation.
21. The process according to any of the preceding embodiments, wherein the monolithic shaped catalyst bodies, based on the overall shaped body, have a greatest dimension in any direction of at least 1 cm, preferably at least 2 cm, especially at least 5 cm.
22. The process according to any of the preceding embodiments, wherein the monolithic shaped catalyst bodies comprise at least one element selected from Ni, Fe, Co, Cu, Cr, Pt, Ag, and Au, preferably selected from the Ni, Co and Cu.
23. The process according to any of the preceding embodiments, wherein the monolithic shaped catalyst bodies are in the form of a foam.
24. The process according to any of the preceding embodiments, wherein the reactor used for hydrogenation comprises a fixed catalyst bed comprising monolithic shaped catalyst bodies or consisting of monolithic shaped catalyst bodies comprising at least one first metal selected from Ni, Fe, Co, Cu, Cr, Pt, Ag, and Au, and comprising at least one second component selected from Al, Zn and Si, wherein the fixed catalyst bed is activated by subjecting it to a treatment with an aqueous base.
25. The process according to embodiment 25, in which
a) a fixed catalyst bed comprising monolithic shaped catalyst bodies or consisting of monolithic shaped catalyst bodies comprising at least one first metal selected from Ni, Fe, Co, Cu, Cr, Pt, Ag, and Au, and comprising at least one second component selected from Al, Zn and Si, is introduced into a reactor,
b) the fixed catalyst bed, for activation, is subjected to a treatment with an aqueous base,
c) the activated fixed catalyst bed obtained in step b) is optionally subjected to a treatment with a wash medium selected from water, C₁-C₄-alkanols and mixtures thereof,
d) the fixed catalyst bed obtained after the activation in step b) or after the treatment in step c) is optionally contacted with a dopant including at least one element other than the first metal and the second component of the shaped catalyst bodies used in step a).
26. The process according to either of embodiments 25 and 26, wherein the shaped catalyst bodies are provided by
a1) providing a shaped metal foam body comprising at least one first metal selected from Ni, Fe, Co, Cu, Cr, Pt, Ag, and Au,
a2) applying at least one second component comprising an element selected from Al, Zn and Si to the surface of the shaped metal foam body, and
a3) forming an alloy by alloying the shaped metal foam body obtained in step a2) at least over part of its surface.

### DESCRIPTION OF THE INVENTION

Hydrogenation

In the context of the invention, hydrogenation is understood quite generally to mean the reaction of an organic compound with addition of H2 onto this compound. Preference is given to hydrogenating functional groups to the correspondingly hydrogenated groups. These include, for example, the hydrogenation of nitro groups, nitroso groups, nitrile groups or imine groups to give amine groups. These further include, for example, the hydrogenation of aromatics to give saturated cyclic compounds. These further include, for example, the hydrogenation of carbon-carbon triple bonds to give double bonds and/or single bonds. These further include, for example, the hydrogenation of carbon-carbon double bonds to give single bonds. These finally include, for example, the hydrogenation of ketones, aldehydes, esters, acids or anhydrides to give alcohols.

Preference is given to the hydrogenation of carbon-carbon triple bonds, carbon-carbon double bonds, aromatic compounds, compounds comprising carbonyl groups, nitriles and nitro compounds. Compounds comprising carbonyl groups suitable for hydrogenation are ketones, aldehydes, acids, esters and anhydrides.

Particular preference is given to the hydrogenation of carbon-carbon triple bonds, carbon-carbon double bonds, nitriles, ketones and aldehydes.

More preferably, the hydrogenatable organic compound is selected from butyne-1,4-diol, butene-1,4-diol, 4-hydroxybutyraldehyde, hydroxypivalic acid, hydroxypivalaldehyde, n- and isobutyraldehyde, n- and isovaleraldehyde, 2-ethylhex-2-enal, 2-ethylhexanal, the isomeric nonanals, cyclododeca-1,5,9-triene, benzene, furan, furfural, phthalic esters, acetophenone and alkyl-substituted acetophenones. Most preferably, the hydrogenatable organic compound is selected from butyne-1,4-diol, butene-1,4-diol, n- and isobutyraldehyde, hydroxypivalaldehyde, 2-ethylhex-2-enal, the isomeric nonanals and 4-isobutylacetophenone.

The hydrogenation of the invention leads to hydrogenated compounds which correspondingly no longer comprise the group to be hydrogenated. If a compound comprises at least two different hydrogenatable groups, it may be desirable to hydrogenate just one of the unsaturated groups, for example when a compound has an aromatic ring and additionally a keto group or an aldehyde group. This includes, for example, the hydrogenation of 4-isobutylacetophenone to 1-(4′-isobutylphenyl)ethanol or the hydrogenation of a C-C-unsaturated ester to the corresponding saturated ester. In principle, simultaneously or instead of a hydrogenation in the context of the invention, an unwanted hydrogenation of other hydrogenatable groups may also occur, for example of carbon-carbon single bonds or of C—OH bonds to water and hydrocarbons. This includes, for example, the hydrogenolysis of butane-1,4-diol to propanal or butanol. These latter hydrogenations generally lead to unwanted by-products and are therefore undesirable. Preferably, the hydrogenation of the invention in the presence of a correspondingly activated catalyst features a high selectivity with respect to the desired hydrogenation reactions. These especially include the hydrogenation of butyne-1,4-diol or butene-1,4-diol to butane-1,4-diol. These further especially include the hydrogenation of n- and isobutyraldehyde to n- and isobutanol. These further especially include the hydrogenation of hydroxypivalaldehyde or of hydroxypivalic acid to neopentyl glycol. These further especially include the hydrogenation of 2-ethylhex-2-enal to 2-ethylhexanol. These further especially include the hydrogenation of nonanals to nonanols. These further especially include the hydrogenation of 4-isobutylacetophenone to 1-(4′-isobutylphenyl)ethanol.

The hydrogenation is preferably conducted continuously.

In the simplest case, the hydrogenation is effected in a single hydrogenation reactor. In a specific execution of the process according to the invention, the hydrogenation is effected in n series-connected hydrogenation reactors, where n is an integer of at least 2. Suitable values of n are 2, 3, 4, 5, 6, 7, 8, 9 and 10. Preferably, n is 2 to 6 and especially 2 or 3. In this execution, the hydrogenation is preferably effected continuously.

The reactors used for hydrogenation may have a fixed catalyst bed formed from identical or different shaped catalyst bodies. The fixed catalyst bed may have one or more reaction zones. Various reaction zones may have shaped catalyst bodies of different chemical composition of the catalytically active species. Various reaction zones may also have shaped catalyst bodies of identical chemical composition of the catalytically active species but in different concentration. If at least 2 reactors are used for hydrogenation, the reactors may be identical or different reactors. These may, for example, each have the same or different mixing characteristics and/or be divided once or more than once by internals.

Suitable pressure-resistant reactors for the hydrogenation are known to those skilled in the art. These include the generally customary reactors for gas-and liquid reactions, for example tubular reactors, shell and tube reactors and gas circulation reactors. A specific embodiment of the tubular reactors is that of shaft reactors.

The process of the invention is conducted in fixed bed mode. Operation in fixed bed mode can be conducted, for example, in liquid phase mode or in trickle mode.

The reactors used for hydrogenation comprise a fixed catalyst bed activated by the process of the invention, through which the reaction medium flows. The fixed catalyst bed may be formed from a single kind of shaped catalyst bodies or from various shaped catalyst bodies. The fixed catalyst bed may have one or more zones, in which case at least one of the zones comprises a material active as a hydrogenation catalyst.

Each zone may have one or more different catalytically active materials and/or one or more different inert materials. Different zones may each have identical or different compositions. It is also possible to provide a plurality of catalytically active zones separated from one another, for example, by inert beds or spacers. The individual zones may also have different catalytic activity. To this end, it is possible to use different catalytically active materials and/or to add an inert material to at least one of the zones. The reaction medium which flows through the fixed catalyst bed comprises at least one liquid phase. The reaction medium may also additionally comprise a gaseous phase.

During the hydrogenation, the CO content in the gas phase within the reactor is preferably within a range from 0.1 to 10 000 ppm by volume, more preferably within a range from 0.15 to 5000 ppm by volume, especially within a range from 0.2 to 1000 ppm by volume. The total CO content within the reactor is composed of the CO in the gas phase and liquid phase, which are in equilibrium with one another. For practical purposes, the CO content is determined in the gas phase and the values reported here relate to the gas phase.

A concentration profile over the reactor is advantageous, and the concentration of CO should rise in flow direction of the reaction medium of the hydrogenation along the reactor.

It has now been found that, surprisingly, a particularly high selectivity is achieved in the hydrogenation when the concentration of CO increases in flow direction of the reaction medium of the hydrogenation reaction and when the fixed catalyst beds comprise shaped catalyst bodies having pores and/or channels, it is preferably the case that, in any section in the normal plane to flow direction through the fixed catalyst bed, at least 90% of the pores and channels, more preferably at least 98% of the pores and channels, have an area of not more than 3 mm². Preferably, the CO content at the exit of the reaction medium from the fixed catalyst bed is at least 5 mol % higher, more preferably at least 25 mol % higher, especially at least 75 mol % higher, than the CO content on entry of the reaction medium into the fixed catalyst bed. To produce a CO gradient in flow direction of the reaction mixture through the fixed catalyst bed, for example, CO can be fed into the fixed catalyst bed at one or more points.

The content of CO will be determined, for example, by means of gas chromatography via taking of individual samples or by online measurement. Preference is given to determination by online measurement. In the taking of samples prior to entry of the reaction medium into the reactor, the procedure is advantageously to take both gas and liquid and to decompress them, in order that formation of an equilibrium between gas and liquid is assured. The content of CO is then determined in the gas phase.

Online measurement can be effected directly in the reactor, for example prior to entry of the reaction medium into the fixed catalyst bed and after exit of the reaction medium from the fixed catalyst bed.

The CO content can be adjusted, for example, by the addition of CO to the hydrogen used for the hydrogenation. Of course, CO can also be fed into the reactor separately from the hydrogen. When the reaction mixture of the hydrogenation is conducted at least partly in a liquid circulation stream, CO can also be fed into this circulation stream. CO can also be formed from components present in the reaction mixture of the hydrogenation, for example as reactants to be hydrogenated or as intermediates or by-products obtained in the hydrogenation. For example, CO can be formed by formic acid, formates or aldehyde present in the reaction mixture of the hydrogenation by decarbonylation. CO can likewise also be formed by decarbonylation of aldehydes other than formaldehyde or by dehydrogenation of primary alcohols to aldehydes and subsequent decarbonylation. These unwanted side reactions include, for example, C-C or C-X scissions, such as propanol formation or butanol formation from butane-1,4-diol. It has also been found that the conversion in the hydrogenation can be only inadequate when the CO content in the gas phase within the reactor is too high, i.e. specifically above 10 000 ppm by volume.

The conversion in the hydrogenation is preferably at least 90 mol %, more preferably at least 95 mol %, particularly at least 99 mol %, especially at least 99.5 mol %, based on the total molar amount of hydrogenatable compounds in the starting material used for hydrogenation. The conversion is based on the amount of the desired target compound obtained, irrespective of how many molar equivalents of hydrogen have been absorbed by the starting compound in order to arrive at the target compound. If a starting compound used in the hydrogenation comprises two or more hydrogenatable groups or comprises a hydrogenatable group that can absorb two or more equivalents of hydrogen (for example an alkyne group), the desired target compound may be the product either of a partial hydrogenation (e.g. alkyne to alkene) or of a full hydrogenation (e.g. alkyne to alkane).

It is important for the success of the hydrogenation of the invention that the reaction mixture of the hydrogenation (i.e. gas and liquid stream) flows very predominantly through the structured catalyst and does not flow past it, as is the case, for example, in conventional random fixed catalyst beds.

Preferably, more than 90% of the stream (i.e. of the sum total of gas and liquid stream) should flow through the fixed catalyst bed, preferably more than 95%, more preferably >99%.

The fixed catalyst beds used in accordance with the invention have, in any section in the normal plane to flow direction (i.e. horizontally) through the fixed catalyst bed, based on the total area of the section, preferably not more than 5%, more preferably not more than 1% and especially not more than 0.1% free area that is not part of the shaped catalyst bodies. The area of the pores and channels that open at the surface of the shaped catalyst bodies is not counted as part of this free area. The figure for free area relates exclusively to sections through the fixed catalyst bed in the region of the shaped catalyst bodies and not any internals such as flow distributors.

When the fixed catalyst beds used in accordance with the invention comprise shaped catalyst bodies having pores and/or channels, it is preferably the case that, in any section in the normal plane to flow direction through the fixed catalyst bed, at least 90% of the pores and channels, more preferably at least 98% of the pores and channels, have an area of not more than 1 mm².

When the fixed catalyst beds used in accordance with the invention comprise shaped catalyst bodies having pores and/or channels, it is preferably the case that, in any section in the normal plane to flow direction through the fixed catalyst bed, at least 90% of the pores and channels, more preferably at least 98% of the pores and channels, have an area of not more than 0.7 mm².

In the fixed catalyst beds of the invention, preferably over at least 90% of the length in flow direction through the fixed catalyst bed, at least 95% of the reactor cross section, more preferably at least 98% of the reactor cross section, especially at least 99% of the reactor cross section, is filled with shaped catalyst bodies.

In order that good mass transfer takes place in the structure catalysts, the velocity with which the reaction mixture flows through the fixed catalyst bed should not be too low. Preferably, the flow velocity of the liquid reaction mixture through the reactor comprising the fixed catalyst bed is at least 30 m/h, preferably at least 50 m/h, especially at least 80 m/h. Preferably, the flow velocity of the liquid reaction mixture through the reactor comprising the fixed catalyst bed is at most 1000 m/h, preferably at most 500 m/h, especially at most 400 m/h.

The flow velocity of the reaction mixture, specifically in the case of an upright reactor, is not of critical significance in principle. The hydrogenation can be effected either in liquid phase mode or trickle mode. Liquid phase mode, wherein the reaction mixture to be hydrogenated is fed in at the liquid phase end of the fixed catalyst bed and is removed at the top end after passing through the fixed catalyst bed, may be advantageous. This is true particularly when the gas velocity should only be low (e.g. <50 m/h). These flow velocities are generally achieved by recycling a portion of the liquid stream leaving the reactor again, combining the recycled stream with the reactant stream either upstream of the reactor or else within the reactor. The reactant stream can also be fed in divided over the length and/or width of the reactor.

In a preferred embodiment, the reaction mixture of the hydrogenation is at least partly conducted in a liquid circulation stream.

The ratio of reaction mixture conducted in the circulation stream to freshly supplied reactant stream is preferably within a range from 1:1 to 1000:1, more preferably from 2:1 to 500:1, especially from 5:1 to 200:1.

Preferably, an output is withdrawn from the reactor and subjected to a gas/liquid separation to obtain a hydrogen-containing gas phase and a product-containing liquid phase. For gas/liquid separation, it is possible to use the apparatuses that are customary for the purpose and are known to those skilled in the art, such as the customary separation vessels (separators). The temperature in the gas/liquid separation is preferably just as high as or lower than the temperature in the reactor. The pressure in the gas/liquid separation is preferably just as high as or lower than the pressure in the reactor. Preferably, the gas/liquid separation is effected essentially at the same pressure as in the reactor. This is the case especially when the liquid phase and optionally the gas phase are conducted in a circulation stream. The pressure differential between reactor and gas/liquid separation is preferably not more than 10 bar, especially not more than 5 bar. It is also possible to configure the gas/liquid separation in two stages. The absolute pressure in the second gas/liquid separation in that case is preferably within a range from 0.1 to 2 bar.

The product-containing liquid phase obtained in the gas/liquid separation is generally at least partly discharged. The product of the hydrogenation can be isolated from this output, optionally after a further workup. In a preferred embodiment, the product-containing liquid phase is at least partly recycled into the hydrogenation as liquid circulation stream.

The hydrogen-containing gas phase obtained in the phase separation can be at least partly discharged as offgas. In addition, the hydrogen-containing gas phase obtained in the phase separation can be at least partly recycled into the hydrogenation. The amount of hydrogen discharged via the gas phase is 0 to 500 mol % of the amount of hydrogen which is consumed in molar terms of hydrogen in the hydrogenation. For example, in the case of consumption of one mole of hydrogen, 5 mol of hydrogen can be discharged as offgas. More preferably, the amount of hydrogen discharged via the gas phase is not more than 100 mol %, especially not more than 50 mol %, of the amount of hydrogen which is consumed in moles of hydrogen in the hydrogenation. By means of this discharge stream, it is possible to control the CO content in the gas phase in the reactor. In a specific execution, the hydrogen-containing gas phase obtained in the phase separation is not recycled. Should this be desired, however, this is preferably up to 1000% of the amount based on the amount of gas required in chemical terms for the conversion, more preferably up to 200%.

The gas loading, expressed in terms of the superficial gas velocity at the reactor exit, is generally not more than 200 m/h, preferably not more than 100 m/h, more preferably not more than 70 m/h, especially not more than 50 m/h. The gas loading consists essentially of hydrogen, preferably to an extent of at least 60% by volume. The gas velocity at the reactor inlet is extremely variable since hydrogen can also be added in intermediate feeds. If all the hydrogen is added at the reactor inlet, the gas velocity is generally higher than at the reactor outlet.

The absolute pressure in the hydrogenation is preferably within a range from 1 to 330 bar, more preferably within a range from 5 to 100 bar, especially within a range from 10 to 60 bar.

The temperature in the hydrogenation is preferably within a range from 40 to 300° C., more preferably from 70 to 220° C., especially from 80 to 200° C.

In a specific execution, the fixed catalyst bed has a temperature gradient during the hydrogenation. Preferably, the temperature differential between the coldest point in the fixed catalyst bed and the warmest point in the fixed catalyst bed is kept at not more than 50 K. Preferably, the temperature differential between the coldest point in the fixed catalyst bed and the warmest point in the fixed catalyst bed is kept within a range from 0.5 to 40 K, preferably within a range of 1 to 30 K.

Catalyst

In the context of the invention, a fixed catalyst bed is understood to mean an apparatus installed into a reactor which is at a fixed location (immobilized) during the hydrogenation, and which comprises one or preferably more than one shaped catalyst body. The fixed catalyst bed is introduced into the reactor by installation of the shaped catalyst bodies at a fixed location. The resulting fixed catalyst bed has a multitude of channels through which the reaction mixture for the hydrogenation reaction flows.

For production of a suitable fixed catalyst bed, the monolithic shaped catalyst bodies can be installed alongside one another and/or one on top of another in the reactor interior. Processes for installation of shaped catalyst bodies are known in principle to the person skilled in the art. For example, one or more layers of a catalyst foam can be introduced into the reactor. Monoliths each consisting of a ceramic block may be stacked alongside one another and one on top of another in the reactor interior. It is essential to the invention that the reaction mixture of the hydrogenation reaction flows exclusively or essentially through the shaped catalyst bodies and not past them. In order to assure flow with minimum bypassing, the monolithic shaped catalyst bodies can be sealed with respect to one another and/or with respect to the inner wall of the reactor by means of suitable devices. These include, for example, sealing rings, sealing mats, etc., consisting of a material inert under the treatment and reaction conditions.

The shaped catalyst bodies are preferably installed into the reactor in one or more essentially horizontal layers with channels which enable flow of the fixed catalyst bed through in flow direction of the aqueous base used for activation and the reaction mixture of the catalyzed reaction. The incorporation is preferably effected in such a way that the fixed catalyst bed very substantially fills the reactor cross section. If desired, the fixed catalyst bed may also comprise further internals such as flow distributors, apparatuses for feeding in gaseous or liquid reactants, measuring elements, especially for temperature measurement, or inert packings.

For the hydrogenation by the process of the invention, suitable reactors in principle are pressure-resistant reactors as customarily used for exothermic heterogeneous reactions involving feeding in one gaseous and one liquid reactant. These include the generally customary reactors for gas-and liquid reactions, for example tubular reactors, shell and tube reactors and gas circulation reactors. A specific embodiment of the tubular reactors is that of shaft reactors. Reactors of this kind are known in principle to the person skilled in the art. More particularly, a cylindrical reactor having a vertical longitudinal axis is used, having, at the base or top of the reactor, an inlet apparatus or a plurality of inlet apparatuses for feeding in a reactant mixture comprising at least one gaseous and at least one liquid component. Substreams of the gaseous and/or the liquid reactant can be fed to the reactor additionally, if desired, via at least one further feed apparatus. The reaction mixture of the hydrogenation generally takes the form of a biphasic mixture having a liquid phase and a gaseous phase. It is also possible that two liquid phases are present as well as the gas phase, for example when further components are present in the hydrogenation.

The processes of the invention are specifically suitable for hydrogenations which are to be conducted on an industrial scale. Preferably, the reactor in that case has an internal volume in the range from 0.1 to 100 m³, preferably from 0.5 to 80 m³. The term “internal volume” relates to the volume including the fixed catalyst bed(s) present in the reactor and any further internals present. The technical advantages associated with the process of the invention are of course also manifested even in reactors with a smaller internal volume.

In the process of the invention, “monolithic” shaped catalyst bodies are used. Monolithic shaped bodies in the context of the invention are structured shaped bodies suitable for production of immobile structured fixed catalyst beds. By contrast with particulate catalysts, it is possible to use monolithic shaped bodies to create essentially coherent and seamless fixed catalyst beds. This corresponds to the definition of monolithic in the sense of “consisting of one piece”. The monolithic shaped catalyst bodies of the invention, by contrast with random catalyst beds, for example composed of pellets, in many cases feature a higher ratio of axial flow (longitudinal flow) to radial flow (crossflow). Monolithic shaped catalyst bodies correspondingly have channels in flow direction of the reaction medium of the hydrogenation reaction. Particulate catalysts display the catalytically active sites generally on an outer surface. Fixed catalyst beds composed of monolithic shape bodies have a multitude of channels, with the catalytically active sites arranged at the surface of the channel walls. The reaction mixture of the hydrogenation reaction can flow through these channels in flow direction through the reactor. Thus, there is generally more intense contacting of the reaction mixture with the catalytically active sites than in the case of random catalyst beds composed of particulate shaped bodies.

The monolithic shaped bodies used in accordance with the invention are not shaped bodies composed of individual catalyst bodies having a greatest longitudinal dimension in any direction of less than 1 cm. Such non-monolithic shaped bodies lead to fixed catalyst beds in the form of standard random catalyst beds. The monolithic shaped catalyst bodies used in accordance with the invention have a regular flat or three-dimensional structure and as such differ from supports in particle form which are used in the form of a random bed.

The monolithic shaped catalyst bodies used in accordance with the invention, based on the overall shaped body, have a smallest dimension in any direction of preferably at least 1 cm, more preferably at least 2 cm, especially at least 5 cm. The maximum value for the greatest dimension in any direction is uncritical in principle and generally results from the production process for the shaped bodies. For example, shaped bodies in the form of foams may be sheetlike structures having a thickness within a range from millimeters to centimeters, a width in the range from a few centimeters to a few hundred centimeters, and a length (as the greatest dimension in any direction) of up to several meters.

The monolithic shaped catalyst bodies used in accordance with the invention, by contrast with bulk materials, can preferably be combined in a form-fitting manner to form larger units or consist of units larger than bulk materials.

The monolithic shaped catalyst bodies used in accordance with the invention generally also differ from particulate catalysts or the supports thereof in that they are present in significantly fewer parts. For instance, in accordance with the invention, a fixed catalyst bed may be used in the form of a single shaped body. In general, however, several shaped bodies are used to produce a fixed catalyst bed. The monolithic shaped catalyst bodies used in accordance with the invention generally have extended three-dimensional structures. The shaped catalyst bodies used in accordance with the invention are generally permeated by continuous channels. The continuous channels may have any geometry; for example, they may be in a honeycomb structure. Suitable shaped catalyst bodies can also be produced by shaping flat support structures, for example by rolling or bending the flat structures to give three-dimensional figures. Proceeding from flat substrates, the outer shape of the shaped bodies can be adapted here in a simple manner to given reactor geometries.

It is a feature of the monolithic shaped catalyst bodies used in accordance with the invention that they can be used to produce fixed catalyst beds where controlled flow through the fixed catalyst bed is possible. Movement of the shaped catalyst bodies under the conditions of the catalyzed reaction, for example mutual friction of the shaped catalyst bodies, is avoided. The ordered structure of the shaped catalyst bodies and the resulting fixed catalyst bed results in improved options for the optimal operation of the fixed catalyst bed in terms of flow methodology.

The monolithic shaped catalyst bodies used in the process of the invention are preferably in the form of a foam, mesh, woven fabric, loop-drawn knitted fabric, loop-formed knitted fabric or another monolith. The term “monolithic catalyst” in the context of the invention also includes catalyst structures known as “honeycomb catalysts”.

In a specific embodiment, the shaped catalyst bodies are in the form of a foam. The shaped catalyst bodies here may have any suitable outer shapes, for example cubic, cuboidal, cylindrical, etc. Suitable woven fabrics can be produced with different weave types, such as plain weave, body weave, Dutch weave, five-shaft satin weave or else other specialty weaves. Also suitable are wire weaves made from weavable metal wires, such as iron, spring steel, brass, phosphor bronze, pure nickel, Monel, aluminum, silver, nickel silver (copper-nickel-zinc alloy), nickel, chromium nickel, chromium steel, nonrusting, acid-resistant and high-temperature-resistant chromium nickel steels, and titanium. The same applies to loop-drawn and loop-formed knitted fabrics. It is likewise possible to use woven fabrics, loop-drawn knitted fabrics or loop-formed knitted fabrics made from inorganic materials, such as from Al₂O₃ and/or SiO₂. Also suitable are woven fabrics, loop-drawn knitted fabrics or loop-formed knitted fabrics made from polymers such as polyamides, polyesters, polyolefins (such as polyethylene, polypropylene), polytetrafluoroethylene, etc. The aforementioned woven fabrics, loop-drawn knitted fabrics or loop-formed knitted fabrics, but also other flat structured catalyst supports, can be shaped to form larger three-dimensional structures, called monoliths. It is likewise possible to construct monoliths not from flat supports but to produce them directly without intermediate stages, for example the ceramic monoliths known to those skilled in the art with flow channels.

Suitable shaped catalyst bodies are those as described, for example, in EP-A 0 068 862, EP-A-0 198 435, EP-A 201 614, EP-A 448 884, EP 0 754 664 A2, DE 433 32 93, EP 2 764 916 A1 and US 2008/0171218 A1.

For instance, EP 0 068 862 describes a monolithic shaped body comprising alternating layers of smooth and corrugated sheets in the form of a roll having channels, and wherein the smooth sheets comprise woven, loop-formingly knitted or loop-drawingly knitted textile materials and the corrugated sheets comprise a mesh material.

EP-A-0 198 435 describes a process for preparing catalysts, in which the active components and the promoters are applied to support materials by vapor deposition under ultrahigh vacuum. Support materials used are support materials of the mesh or fabric type. The catalyst fabrics that have been subjected to vapor deposition, for installation into the reactor, are combined to form “catalyst packages” and the shaping of the catalyst packages is adapted to the flow conditions in the reactor.

Suitable processes for vapor deposition and “sputtering deposition” of metals under reduced pressure are known.

The shaped catalyst bodies preferably comprise at least one element selected from Ni, Fe, Co, Cu, Cr, Pt, Ag, Au and Pd. In a specific embodiment, the shaped catalyst bodies comprise Ni. In a specific embodiment, the shaped catalyst bodies do not comprise any palladium. This is understood to mean that, for production of the shaped catalyst bodies, no palladium is actively added, either as catalytically active metal or as promoter element or for provision of the shaped bodies which serve as support material.

Preferably, the shaped catalyst bodies are a Raney metal catalyst.

Preferably, the reactor used for hydrogenation comprises a fixed catalyst bed comprising monolithic shaped catalyst bodies or consisting of monolithic shaped catalyst bodies comprising at least one first metal selected from Ni, Fe, Co, Cu, Cr, Pt, Ag, and Au, and comprising at least one second component selected from Al, Zn and Si, wherein the fixed catalyst bed is activated by subjecting it to a treatment with an aqueous base.

A preferred embodiment is a process in which
a) a fixed catalyst bed comprising monolithic shaped catalyst bodies or consisting of monolithic shaped catalyst bodies comprising at least one first metal selected from Ni, Fe, Co, Cu, Cr, Pt, Ag, and Au, and comprising at least one second component selected from Al, Zn and Si, is introduced into a reactor,
b) the fixed catalyst bed, for activation, is subjected to a treatment with an aqueous base,
c) the activated fixed catalyst bed obtained in step b) is optionally subjected to a treatment with a wash medium selected from water, C₁-C₄-alkanols and mixtures thereof,
d) the fixed catalyst bed obtained after the activation in step b) or after the treatment in step c) is optionally contacted with a dopant including at least one element other than the first metal and the second component of the shaped catalyst bodies used in step a).

More preferably, the monolithic shaped catalyst bodies are in the form of a foam. Suitable in principle are metal foams having various morphological properties in terms of pore size and shape, layer thickness, areal density, geometric surface area, porosity, etc. The production can be effected in a manner known per se. For example, a foam composed of an organic polymer can be coated with at least one first metal and then the polymer can be removed, for example by pyrolysis or dissolution in a suitable solvent, to obtain a metal foam. For coating with at least one first metal or a precursor thereof, the foam composed of the organic polymer can be contacted with a solution or suspension comprising the first metal. This can be effected, for example, by spraying or dipping. Another possibility is deposition by means of chemical vapor deposition (CVD). For example, it is possible to coat a polyurethane foam with the first metal and then pyrolyze the polyurethane foam. A polymer foam suitable for production of shaped catalyst bodies in the form of a foam preferably has a pore size in the range from 100 to 5000 pm, more preferably from 450 to 4000 pm and especially from 450 to 3000 pm. A suitable polymer foam preferably has a layer thickness of 5 to 60 mm, more preferably of 10 to 30 mm. A suitable polymer foam preferably has a density of 300 to 1200 kg/m³. The specific surface area is preferably within a range from 100 to 20 000 m²/m³, more preferably 1000 to 6000 m²/m³. The porosity is preferably within a range from 0.50 to 0.95.

The second component can be applied in various ways, for example by contacting the shaped body obtained from the first component with the second component by rolling or dipping, or applying the second component by spraying, scattering or pouring. For this purpose, the second material may be in liquid form or preferably in the form of a powder. Another possibility is the application of salts of the second component and subsequent reduction. Another possibility is application of the second component in combination with an organic binder. The production of an alloy on the surface of the shaped body is effected by heating to the alloying temperature. It is possible via the alloying conditions, as explained above, to control the leaching properties of the alloy. When Al is used as the second component, the alloying temperature is preferably within a range from 650 to 1000° C., more preferably 660 to 950° C. When an Ni/Al powder is used as the second component, the alloying temperature is preferably within a range from 850 to 900° C., more preferably 880 to 900° C. It may be advantageous, during the alloying, to continuously raise the temperature and then keep it at the maximum value for a period of time. Subsequently, the coated and heated shaped foam catalyst bodies can be cooled down.

In a preferred embodiment, for provision of the shaped catalyst bodies:
a1) a shaped metal foam body comprising at least one first metal selected from Ni, Fe, Co, Cu, Cr, Pt, Ag, and Au is provided,
a2) at least one second component comprising an element selected from Al, Zn and Si is applied to the surface of the shaped metal foam body, and
a3) an alloy by alloying the shaped metal foam body obtained in step a2) is formed at least over part of its surface.

Shaped catalyst bodies of this kind and processes for preparation thereof are described in EP 2 764 916 A1, which is fully incorporated by reference.

Suitable alloying conditions are apparent from the phase diagram of the metals involved, for example the phase diagram of Ni and Al. In this way, for example, it is possible to control the proportion of Al-rich and leachable components, such as NiAl₃ and Ni₂Al₃. The shaped catalyst bodies may comprise dopants in addition to the first and second components. These include, for example, Mn, V, Ta, Ti, W, Mo, Re, Ge, Sn, Sb or Bi.

Preference is given to shaped catalyst bodies in which the first metal comprises Ni or consists of Ni. Preference is further given to shaped catalyst bodies in which the second component comprises Al or consists of Al. A specific embodiment is that of shaped catalyst bodies comprising nickel and aluminum.

For the production of a monolithic shaped catalyst body in the form of a foam, preference is given to using an aluminum powder having a particle size of at least 5 μm. Preferably, the aluminum powder has a particle size of not more than 75 μm.

Preferably, for the production of a monolithic shaped catalyst body in the form of a foam,
a1) a shaped metal foam body comprising Ni is provided,
a2) an aluminum-containing suspension in a solvent is applied to the surface of the shaped metal foam body,
a3) an alloy by alloying the shaped metal foam body obtained in step a2) is formed at least over part of its surface.

More preferably, the aluminum-containing suspension additionally comprises polyvinylpyrrolidone. The amount of the polyvinylpyrrolidone is preferably 0.1% to 5% by weight, more preferably 0.5% to 3% by weight, based on the total weight of the aluminum-containing suspension. The molecular weight of the polyvinylpyrrolidone is preferably within a range from 10 000 to 1 300 000 g/mol.

More preferably, the aluminum-containing suspension comprises a solvent selected from water, ethylene glycol and mixtures thereof.

The alloy is preferably formed in the course of stepwise heating in the presence of a gas mixture comprising hydrogen and at least one gas which is inert under the reaction conditions. The inert gas used is preferably nitrogen. An example of a suitable gas mixture is one comprising 50% by volume of N₂ and 50% by volume of H₂. The alloy can be formed, for example, in a rotary kiln. Suitable heating rates are within a range from 1 to 10 K/nnin, preferably 3 to 6 K/min. It may be advantageous to keep the temperature essentially constant (isothermal) once or more than once for a particular period of time during the heating. For example, during the heating, the temperature may be kept constant at about 300° C., about 600° C. and/or about 700° C. The period of time over which the temperature is kept constant is preferably about 1 to 120 minutes, more preferably 5 to 60 minutes. Preferably, during the heating, the temperature is kept constant within a range from 650 to 920° C. When the temperature is kept constant on multiple occasions, the last stage is preferably within a range from 650 to 920° C. The alloy is further preferably formed in the course of stepwise cooling. Preferably, the cooling is effected down to a temperature in the range from 150 to 250° C. in the presence of a gas mixture comprising hydrogen and at least one gas which is inert under the reaction conditions. The inert gas used is preferably nitrogen. An example of a suitable gas mixture is one comprising 50% by volume of N₂ and 50% by volume of H₂. Preferably, the further cooling is effected in the presence of at least one inert gas, preferably in the presence of nitrogen.

Preferably, the weight of the monolithic shaped catalyst body in the form of a foam is 35% to 60%, more preferably 40% to 50%, higher than the weight of the shaped metal foam body used for preparation thereof.

Preferably, the intermetallic phases thus obtained on the support metal framework consist mainly of Ni₂Al₃ and NiAl₃.

Activation (step b))

Preferably, the shaped catalyst bodies used for activation, based on the total weight, have 60% to 95% by weight, more preferably 70% of 80% by weight, of a first metal selected from Ni, Fe, Co, Cu, Cr, Pt, Ag, Au, Mn, Re, Ru, Rh and Ir.

More preferably, the shaped catalyst bodies used for activation, based on the total weight, have 60% to 95% by weight, especially 70% of 80% by weight, of a first metal selected from Ni, Fe, Co, Cu, Cr, Pt, Ag, and Au.

Preferably, the shaped catalyst bodies used for activation, based on the total weight, have 5% to 40% by weight, more preferably 20% of 30% by weight, of a second component selected from Al, Zn and Si.

Preferably, the shaped catalyst bodies used for activation, based on the total weight, have 60% to 95% by weight, more preferably 70% of 80% by weight, of Ni.

Preferably, the shaped catalyst bodies used for activation, based on the total weight, have 5% to 40% by weight, more preferably 20% of 30% by weight, of Al.

During the activation, the fixed catalyst bed is subjected to a treatment with an aqueous base as treatment medium, wherein the second (leachable) component of the shaped catalyst bodies is at least partly dissolved and removed from the shaped catalyst bodies. This treatment with the aqueous base proceeds exothermically, such that the fixed catalyst bed is heated as a result of the activation. The heating of the fixed catalyst bed is dependent on the concentration of the aqueous base used. If no heat is removed from the reactor by active cooling and it is instead transferred to the treatment medium such that an adiabatic mode of operation is implemented to a certain degree, a temperature gradient forms in the fixed catalyst bed during the activation, with increasing temperature in flow direction of the aqueous base. But when heat is removed from the reactor by active cooling, a temperature gradient forms in the fixed catalyst bed during the activation.

Preferably, the activation removes 30% to 70% by weight, more preferably 40% to 60% by weight, of the second component from the shaped catalyst bodies, based on the original weight of the second component.

Preferably, the shaped catalyst bodies used for activation comprise Ni and Al, and the activation removes 30% to 70% by weight, more preferably 40% to 60% by weight, of the Al, based on the original weight.

The amount of the second component, for example aluminum, leached out of the shaped catalyst bodies can be determined, for example, via elemental analysis, by determining the content of the second component in the total amount of the laden aqueous base discharged and the wash medium. Alternatively, the determination of the amount of the second component leached out of the shaped catalyst bodies can be determined via the amount of hydrogen formed in the course of activation. If aluminum is used, the leaching-out of 2 mol of aluminum results in production of 3 mol of hydrogen in each case.

The activation of the fixed catalyst bed can be effected in liquid phase mode or trickle mode. Preference is given to liquid phase mode, wherein the fresh aqueous base is fed in on the liquid phase side of the fixed catalyst bed and, after passing through the fixed catalyst bed, is withdrawn at the top end.

After passing through the fixed catalyst bed, a laden aqueous base is obtained. The laden aqueous base has a lower concentration of base compared to the aqueous base prior to passage through the fixed catalyst bed and is enriched in the reaction products that have formed in the activation and are at least partly soluble in the base. These reaction products include, for example, in the case of use of aluminum as the second (leachable) component, alkali metal aluminates, aluminum hydroxide hydrates, hydrogen, etc. (see, for example, U.S. Pat. No. 2,950,260).

The statement that the fixed catalyst bed has a temperature gradient during the activation is understood in the context of the invention such that the fixed catalyst bed has this temperature gradient over a relatively long period of time in the overall activation. Preferably, the fixed catalyst bed has a temperature gradient until at least 50% by weight, preferably at least 70% by weight, especially at least 90% by weight, of the amount of aluminum to be removed from the shaped catalyst bodies has been removed. If the strength of the aqueous base used is not increased over the course of the activation and/or the temperature of the fixed catalyst bed is increased as a result of a lesser degree of cooling than at the start of the activation or external heating, the temperature differential between the coldest point in the fixed catalyst bed and the warmest point in the fixed catalyst bed will become increasingly smaller over the course of the activation and may then even assume the value of zero toward the end of the activation.

Preferably, the temperature differential between the coldest point in the fixed catalyst bed and the warmest point in the fixed catalyst bed is kept at not more than 50 K. To determine the temperature differential over the course of the fixed catalyst bed, it can be provided with customary measurement units for temperature measurement. To determine the temperature differential between the warmest point in the fixed catalyst bed and the coldest point in the fixed catalyst bed, in the case of a reactor without active cooling, it is generally sufficient to determine the temperature differential between the furthest point upstream in the fixed catalyst bed and the furthest point downstream in the fixed catalyst bed. In the case of an actively cooled reactor, it may be advisable to provide at least one further temperature sensor (for example 1, 2 or 3 further temperature sensor(s)) between the furthest point upstream in the fixed catalyst bed and the furthest point downstream in the fixed catalyst bed.

More preferably, the temperature differential between the coldest point in the fixed catalyst bed and the warmest point in the fixed catalyst bed is kept at not more than 40 K, especially at not more than 25 K.

Preferably, the temperature differential between the coldest point in the fixed catalyst bed and the warmest point in the fixed catalyst bed at the start of activation is kept within a range from 0.1 to 50 K, preferably within a range from 0.5 to 40 K, especially within a range from 1 to 25 K. It is possible, at the start of the activation, first to initially charge an aqueous medium without base and then to feed in fresh base until the desired concentration has been attained. In this case, the temperature differential between the coldest point in the fixed catalyst bed and the warmest point in the fixed catalyst bed at the start of activation is understood to mean the juncture when the desired base concentration has been attained for the first time at the reactor entrance.

The parameter of the temperature gradient in the fixed catalyst bed can be controlled in a reactor without active cooling by choosing the amount and concentration of the aqueous base fed in according to the capacity of the medium used for activation. To control the parameter of the temperature gradient in the fixed catalyst bed in a reactor with active cooling, heat is removed by heat exchange in addition to the medium used for activation. Such removal of heat can be effected by cooling the medium used for activation in the reactor used and/or, if present, the liquid circulation stream.

Preferably, the monolithic shaped catalyst bodies are activated by subjecting them to a treatment with an aqueous base having a strength of not more than 3.5% by weight. Particular preference is given to the use of an aqueous base having a maximum strength of 3.0% by weight. Preferably, the shaped catalyst bodies, for activation, are subjected to a treatment with an aqueous base having a strength of 0.1% to 3.5% by weight, more preferably an aqueous base having a strength of 0.5% to 3.5% by weight. The concentration figure is based on the aqueous base prior to contact thereof with the shaped catalyst bodies. If the aqueous base is contacted just once with the shaped catalyst bodies for activation, the concentration figure is based on the fresh aqueous base. If the aqueous base is conducted at least partly in a liquid circulation stream for activation, fresh base can be added to the laden base obtained after the activation before it is reused for activation of the shaped catalyst bodies. In this context, the concentration values stated above apply analogously.

Compliance with the above-specified concentrations for the aqueous base affords shaped catalyst bodies of Raney metal catalysts having high activity and very good stability. This is especially true of the activation of fixed catalyst beds for hydrogenation reactions on an industrial scale. Surprisingly, the stated concentration ranges for the base are effective in avoiding an excessive temperature increase and the uncontrolled formation of hydrogen in the activation of the catalysts. This advantage is especially effective in reactors on the industrial scale.

In a preferred embodiment, the aqueous base used for activation is at least partly conducted in a liquid circulation stream. In a first embodiment, the reactor is operated in liquid phase mode with the catalyst to be activated. In that case, in a vertically aligned reactor, the aqueous base is fed into the reactor at the liquid phase end and conducted from the bottom upward through the fixed catalyst bed, and an output is removed above the fixed catalyst bed and recycled into the reactor at the liquid phase end. The discharged stream will preferably be subjected here to a workup, for example by removal of hydrogen and/or the discharge of a portion of the laden aqueous phase. In a second embodiment, the reactor is operated in trickle mode with the catalyst to be activated. In that case, in a vertically aligned reactor, the aqueous base is fed into the reactor at the top end and conducted from the top downward through the fixed catalyst bed, and an output is removed below the fixed catalyst bed and recycled into the reactor at the top end. The discharged stream is preferably again subjected here to a workup, for example by removal of hydrogen and/or the discharge of a portion of the laden aqueous phase. Preferably, the activation is effected in a vertical reactor in liquid phase mode (i.e. with a stream directed upward through the fixed catalyst bed). Such a mode of operation is advantageous when the formation of hydrogen during the activation also produces a low gas hourly space velocity, since it can be more easily removed overhead.

In a preferred embodiment, in addition to the base conducted in the liquid circulation stream, the fixed catalyst bed is supplied with fresh aqueous base. Fresh base can be fed into the liquid circulation stream or separately therefrom into the reactor. The fresh aqueous base may also have a higher concentration than 3.5% by weight if the base concentration after the mixing with the recycled aqueous base is not higher than 3.5% by weight.

The ratio of aqueous base conducted in the circulation stream to freshly supplied aqueous base is preferably within a range from 1:1 to 1000:1, more preferably from 2:1 to 500:1, especially from 5:1 to 200:1.

Preferably, the feed rate of the aqueous base (when the aqueous base used for activation is not being conducted in a liquid circulation stream) is not more than 5 L/min per liter of fixed catalyst bed, preferably not more than 1.5 L/min per liter of fixed catalyst bed, more preferably not more than 1 L/min per liter of fixed catalyst bed, based on the total volume of the fixed catalyst bed.

Preferably, the aqueous base used for activation is conducted at least partly in a liquid circulation stream and the feed rate of the freshly supplied aqueous base is not more than 5 L/min per liter of fixed catalyst bed, preferably not more than 1.5 L/min per liter of fixed catalyst bed, more preferably not more than 1 L/min per liter of fixed catalyst bed, based on the total volume of the fixed catalyst bed.

Preferably, the feed rate of the aqueous base (when the aqueous base used for activation is not being conducted in a liquid circulation stream) is within a range from 0.05 to 5 L/min per liter of fixed catalyst bed, more preferably within a range from 0.1 to 1.5 L/min per liter of fixed catalyst bed, especially within a range from 0.1 to 1 L/min per liter of fixed catalyst bed, based on the total volume of the fixed catalyst bed.

Preferably, the aqueous base used for activation is conducted at least partly in a liquid circulation stream and the feed rate of the freshly supplied aqueous base is within a range from 0.05 to 5 L/min per liter of fixed catalyst bed, more preferably within a range from 0.1 to 1.5 L/min per liter of fixed catalyst bed, especially within a range from 0.1 to 1 L/min per liter of fixed catalyst bed, based on the total volume of the fixed catalyst bed.

The control of the feed rate of the fresh aqueous base is an effective way of keeping the temperature gradient that results in the fixed catalyst bed within the desired range of values.

The flow velocity of the aqueous base through the reactor comprising the fixed catalyst bed is preferably at least 0.5 m/h, more preferably at least 3 m/h, especially at least 5 m/h, specifically at least 10 m/h.

In order to avoid mechanical stress on and abrasion of the newly formed porous catalyst metal, it may be advisable not to choose too high a flow velocity. The flow velocity of the aqueous base through the reactor comprising the fixed catalyst bed is preferably not more than 100 m/h, more preferably not more than 50 m/h, especially not more than 40 m/h.

The above-specified flow velocities can be achieved particularly efficiently when at least a portion of the aqueous base is conducted in a liquid circulation stream.

The base used for activation of the fixed catalyst bed is selected from alkali metal hydroxides, alkaline earth metal hydroxides and mixtures thereof. The base is preferably selected from NaOH, KOH and mixtures thereof. Specifically, the base used is NaOH. The base is used for activation in the form of an aqueous solution.

The procedure described above enables effective minimization of leaching of the catalytically active metal, such as nickel, during the activation. A suitable measure of the effectiveness of the activation and the stability of the Raney metal catalyst obtained is the metal content in the laden aqueous phase. In the case of use of a liquid circulation stream, the metal content in the circulation stream is a suitable measure of the effectiveness of the activation and the stability of the Raney metal catalyst obtained. Preferably, the nickel content during the activation in the laden aqueous base or, when the a liquid circulation stream is used for activation, in the circulation stream is not more than 0.1% by weight, more preferably not more than 100 ppm by weight, especially not more than 10 ppm by weight. The nickel content can be determined by means of elemental analysis. The same advantageous values are generally also achieved in the downstream process steps, such as the treatment of the activated fixed catalyst bed with a wash medium, the treatment of the fixed catalyst bed with a dopant, and the use in a hydrogenation reaction.

The process of the invention enables homogeneous distribution of the catalytically active Raney metal over the shaped bodies used and, overall, over the activated fixed catalyst bed used. Only a slight gradient, if any, forms with respect to the distribution of the catalytically active Raney metal in flow direction of the activation medium through the fixed catalyst bed. In other words, the concentration of catalytically active sites upstream of the fixed catalyst bed is essentially equal to the concentration of catalytically active sites downstream of the fixed catalyst bed. This advantageous effect is achieved especially when the aqueous base used for activation is at least partly conducted in a liquid circulation stream. The processes of the invention also enable homogeneous distribution of the second component that has been leached out, for example the aluminum, over the shaped bodies used and, overall, over the activated fixed catalyst bed obtained. Only a slight gradient, if any, forms with respect to the distribution of the second component that has been leached out in flow direction of the activation medium through the fixed catalyst bed.

A further advantage, when the aqueous base used for activation is at least partly conducted in a liquid circulation stream, is that the use amount of aqueous base required can be distinctly reduced. Thus, a straight pass of the aqueous base (without recycling) and the subsequent discharge of the laden base leads to a high demand for fresh base. The supply of suitable amounts of fresh base to the recycle stream ensures that sufficient base for the activation reaction is always present. For this purpose, distinctly smaller amounts are required overall.

After passage through the fixed catalyst bed, a laden aqueous base is obtained, having a lower base concentration compared to the aqueous base prior to passage through the fixed catalyst bed and enriched in the reaction products that are formed in the activation and are at least partly soluble in the base. Preferably, at least a portion of the laden aqueous base is discharged. It is thus possible, even if a portion of the aqueous base is conducted in a circulation stream, to avoid excessive dilution and accumulation of unwanted impurities in the aqueous base used for activation. Preferably, the amount of fresh aqueous base fed in per unit time corresponds to the amount of laden aqueous base discharged.

Preferably, an output of laden aqueous base is withdrawn and subjected to a gas/liquid separation to obtain a hydrogen-containing gas phase and a liquid phase. For gas/liquid separation, it is possible to use the apparatuses that are customary for the purpose are known to those skilled in the art, such as the customary separation vessels. The hydrogen-containing gas phase obtained in the phase separation can be discharged from the process and sent, for example, to thermal utilization. The liquid phase obtained in the phase separation, comprising the laden aqueous base output, is preferably at least partly recycled into the activation as liquid circulation stream. Preferably, a portion of the liquid phase obtained in the phase separation, comprising the laden aqueous base output, is discharged. It is thus possible, as described above, to avoid excessive dilution and accumulation of unwanted impurities in the aqueous base used for activation.

To control the progress of the activation and to determine the amount of the second component, for example aluminum, leached out of the shaped catalyst bodies, it is possible to determine the amount of hydrogen formed in the course of activation. If aluminum is used, the leaching-out of 2 mol of aluminum results in production of 3 mol of hydrogen in each case.

Preferably, the activation of the invention is effected at a temperature of not more than 50° C., preferably at a temperature of not more than 40° C.

Preferably, the activation of the invention is effected at a pressure in the range from 0.1 to 10 bar, more preferably from 0.5 to 5 bar, specifically at ambient pressure.

Treatment with a Wash Medium (Step c))

In the optional step c) of the process of the invention, the activated fixed catalyst bed obtained in step b) is subjected to a treatment with a wash medium selected from water, C₁-C₄-alkanols and mixtures thereof.

Suitable C₁-C₄-alkanols are methanol, ethanol, n-propanol, isopropanol, n-butanol and isobutanol.

Preferably, the wash medium used in step c) comprises water or consists of water.

Preferably, in step c), the treatment with the wash medium is conducted until the wash medium effluent has a conductivity at 20° C. of not more than 200 mS/cm, more preferably of not more than 100 mS/cm, especially of not more than 10 mS/cm.

Preferably, in step c), water is used as wash medium and the treatment with the wash medium is conducted until the wash medium effluent has a pH at 20° C. of not more than 9, preferably of not more than 8, especially of not more than 7.

Preferably, in step c), the treatment with the wash medium is conducted until the wash medium effluent has an aluminum content of not more than 5% by weight, more preferably of not more than 5000 ppm by weight, especially of not more than 500 ppm by weight.

Preferably, in step c), the treatment with the wash medium is conducted at a temperature in the range from 20 to 100° C., more preferably from 20 to 80° C., especially from 25 to 70° C.

Doping (Step d))

Doping refers to the introduction of extraneous atoms into a layer or into the base material of a catalyst. The amount introduced in this operation is generally small compared to the rest of the catalyst material. The doping alters the properties of the starting material in a controlled manner.

In a specific embodiment, the fixed catalyst bed obtained after the activation (i.e. after step b)) and optionally after the treatment with a wash medium (i.e. also after step c), if it is conducted) is contacted with a dopant including at least one element other than the first metal and the second component of the shaped catalyst bodies used in step a). Such elements are referred to hereinafter as “promoter elements”. Preferably, the contacting with the dopant is effected during and/or after the treatment of the activated fixed catalyst bed with a wash medium (i.e. during and/or after step c)).

The dopant preferably comprises at least one promoter element selected from Ti, Ta, Zr, V, Cr, Mo, W, Mn, Re, Fe, Ru, Co, Rh, Ir, Ni, Pd, Pt, Cu, Ag, Au, Ce and Bi.

It is possible that the dopant comprises at least one promoter element which simultaneously fulfills the definition of a first metal in the context of the invention. Promoter elements of this kind are selected from Ni, Fe, Co, Cu, Cr, Mn, Re, Ru, Rh, Ir, Pt, Ag, Au and Pd. In this case, the monolithic shaped body, based on the reduced metal form, contains a majority (i.e. more than 50% by weight) of the first metal and a minority (i.e. less than 50% by weight) of a different metal as dopant. In stating the total amount of the first metal that the monolithic shaped catalyst body comprises, however, all metals that fulfill the definition of a first metal in the context of the invention are calculated with their full proportion by weight (irrespective of whether they act as hydrogenation-active component or as promoter).

In a specific embodiment, the dopant does not comprise any promoter element that fulfills the definition of a first metal in the context of the invention. Preferably, the dopant in that case comprises exclusively a promoter element or more than one promoter element selected from Ti, Ta, Zr, Ce, V, Mo, W and Bi.

Preferably, the dopant comprises Mo as promoter element. In a specific embodiment, the dopant comprises Mo as the sole promoter element.

More preferably, the promoter elements for doping are used in the form of their salts. Suitable salts are, for example, the nitrates, sulfates, acetates, formates, fluorides, chlorides, bromides, iodides, oxides or carbonates. The promoter elements separate of their own accord in their metallic form either because of their baser character compared to Ni or can be reduced to their metallic form by contacting with a reducing agent, for example hydrogen, hydrazine, hydroxylamine, etc. If the promoter elements are added during the activation operation, they may also be present in their metallic form. In this case, it may be advisable for formation of metal-metal compounds to subject the fixed catalyst bed, after the incorporation of the promoter metals, first to an oxidative treatment and then to a reductive treatment.

In a specific embodiment, the fixed catalyst bed is contacted with a dopant comprising Mo as promoter element during and/or after the treatment with a wash medium in step c). Even more specifically, the dopant comprises Mo as the sole promoter element. Suitable molybdenum compounds are selected from molybdenum trioxide, the nitrates, sulfates, carbonates, chlorides, iodides and bromides of molybdenum, and the molybdates. Preference is given to the use of ammonium molybdate. In a preferred embodiment, a molybdenum compound having good water solubility is used. A good water solubility is understood to mean a solubility of at least 20 g/L at 20° C. In the case of use of molybdenum compounds having lower water solubility, it may be advisable to filter the solution prior to the use thereof as dopant. Suitable solvents for doping are water, polar solvents other than water that are inert with respect to the catalyst under the doping conditions, and mixtures thereof. Preferably, the solvent used for doping are selected from water, methanol, ethanol, n-propanol, isopropanol, n-butanol, isobutanol and mixtures thereof.

Preferably, the temperature in the doping is within a range from 10 to 100° C., more preferably from 20 to 60° C., especially from 20 to 40° C.

The concentration of the promoter element in the dopant is preferably within a range from about 20 g/L up to the maximum possible amount of the dopant under the doping conditions. In general, the maximum amount used as a starting point will be a solution saturated at ambient temperature.

The duration of doping is preferably 0.5 to 24 hours.

It may be advantageous that the doping is effected in the presence of an inert gas. Suitable inert gases are, for example, nitrogen or argon.

In a specific embodiment, for doping of shaped catalyst foam bodies, a molybdenum source is dissolved in water and this solution is passed through the previously activated foam. In the case of use of hydrates of ammonium molybdate, for example (NH₄)₆Mo₇O₂₄×4H₂O, the latter is dissolved in water and this solution is used. The usable amount depends greatly on the solubility of the ammonium molybdate and is not critical in principle. For practical purposes, less than 430 g of ammonium molybdate are dissolved per liter of water at room temperature (20° C.). If the doping is conducted at higher temperature than room temperature, it is also possible to use greater amounts. The ammonium molybdate solution is subsequently passed through the activated and washed foam at a temperature of 20 to 100° C., preferably at a temperature of 20 to 40° C. The duration of treatment is preferably 0.5 to 24 h, more preferably 1 to 5 h. In a specific execution, the contacting is effected in the presence of an inert gas, such as nitrogen. The pressure is preferably within a range from 1 to 50 bar, specifically about 1 bar absolute. Thereafter, the doped Raney nickel foam can be used for the hydrogenation either without further workup or after another wash.

The doped shaped catalyst bodies comprise preferably 0.01% to 10% by weight, more preferably 0.1% to 5% by weight, of promoter elements based on the reduced metallic form of the promoter elements and the total weight of the shaped catalyst bodies.

The fixed catalyst bed may comprise the promoter elements in essentially homogeneous or heterogeneous distribution with respect to the concentration thereof. In a specific embodiment, the fixed catalyst bed has a gradient with respect to the concentration of the promoter elements in flow direction. More particularly, the fixed catalyst bed comprises or consists of shaped Ni/Al catalyst bodies which are activated by the process of the invention and/or are doped with Mo, and has a gradient with respect to the Mo concentration in flow direction.

It is possible to obtain a fixed bed catalyst installed in a fixed position in a reactor, and comprising at least one promoter element in essentially homogeneous distribution of its concentration, i.e. not in the form of a gradient. For provision of such a fixed bed catalyst, it is possible to dope the catalyst not in installed form in the fixed bed reactor itself, optionally with circulation, which can give rise to a concentration gradient. Preferably, the doping in that case is effected in an external vessel without circulation and having infinite backmixing, for example a batch reactor without continuous input and output. On completion of doping and optionally washing, such catalysts can be installed in a fixed bed reactor with or without circulation and are thus present without gradients.

For provision of a fixed catalyst bed having a gradient in flow direction with respect to the concentration of the promoter elements, the procedure may be to pass a liquid stream of the dopant through the fixed catalyst bed. If the reactor has a circulation stream, it is alternatively or additionally possible to feed the dopant into the circulation stream in liquid form. In the case of such a procedure, a concentration gradient of the promoter elements in flow direction forms over the entire length of the fixed catalyst bed. If a decrease in the concentration of the promoter element in flow direction of the reaction medium of the reaction to be catalyzed is desired, the liquid stream of the dopant is passed through the fixed catalyst bed in the same direction as the reaction medium of the reaction to be catalyzed. If an increase in the concentration of the promoter element in flow direction of the reaction medium of the reaction to be catalyzed is desired, the liquid stream of the dopant is passed through the fixed catalyst bed in the opposite direction to the reaction medium of the reaction to be catalyzed.

In a first preferred embodiment, the fixed catalyst bed obtained by the process of the invention or a reactor comprising such a fixed catalyst bed serves for hydrogenation of butyne-1,4-diol to obtain butane-1,4-diol. It has now been found that, surprisingly, in the hydrogenation, a particularly high selectivity is achieved when a fixed catalyst bed composed of shaped Ni/Al catalyst bodies which are activated by means of the process of the invention and/or are doped with Mo is used, wherein the concentration of molybdenum increases in flow direction of the reaction medium of the hydrogenation reaction. Preferably, the molybdenum content of the shaped catalyst bodies at the entrance of the reaction medium into the fixed catalyst bed is 0% to 3% by weight, more preferably 0.05% to 2.5% by weight, especially 0.1% to 2% by weight, based on metallic molybdenum and the total weight of the shaped catalyst bodies. Preferably, the molybdenum content of the shaped catalyst bodies at the exit of the reaction medium from the fixed catalyst bed is 0.1% to 10% by weight, more preferably 0.1% to 7% by weight, especially 0.2% to 6% by weight, based on metallic molybdenum and the total weight of the shaped catalyst bodies.

In a second preferred embodiment, the fixed catalyst bed obtained by the process of the invention or a reactor comprising such a fixed catalyst bed serves for hydrogenation of butyraldehyde to obtain n-butanol. It has now been found that, surprisingly, in the hydrogenation, a particularly high selectivity is achieved when a fixed catalyst bed composed of shaped Ni/AI catalyst bodies which are activated by means of the process of the invention and/or are doped with Mo is used, wherein the concentration of molybdenum decreases in flow direction of the reaction medium of the hydrogenation reaction. Preferably, the molybdenum content of the shaped catalyst bodies at the entrance of the reaction medium into the fixed catalyst bed is 0.5% to 10% by weight, more preferably 1% to 9% by weight, especially 1% to 7% by weight, based on metallic molybdenum and the total weight of the shaped catalyst bodies. Preferably, the molybdenum content of the shaped catalyst bodies at the exit of the reaction medium from the fixed catalyst bed is 0% to 7% by weight, more preferably 0.05% to 5% by weight, especially 0.1% to 4.5% by weight, based on metallic molybdenum and the total weight of the shaped catalyst bodies.

It has been found that it is advantageous for the efficiency of the doping of Raney metal catalysts and specifically of Raney metal catalysts having a promoter element, specifically Mo, when the activated fixed catalyst bed, after the activation and before the doping, is subjected to a treatment with a wash medium. This is especially true when Raney nickel catalyst foams are used for the doping. It has especially been found that the adsorption of the molybdenum onto the shaped catalyst bodies is incomplete when, after activation, the content of aluminum that can be washed out is still too high. Preferably, therefore, before the doping in step d), the treatment with a wash medium is conducted in step c) until the wash medium effluent at a temperature of 20° C. has a conductivity of not more than 200 mS/cm. Preferably, in step c), the treatment with the wash medium is conducted until the wash medium effluent has an aluminum content of not more than 500 ppm by weight.

The activated fixed catalyst beds obtained by the process of the invention, optionally comprising dope shaped catalyst bodies, generally feature high mechanical stability and long service lives. Nevertheless, the fixed bed catalyst is mechanically stressed when the components to be hydrogenated flow through it in the liquid phase. This can result in wear or the abrasion of the outer layers of the active catalyst species in the long term. If the Raney nickel foam catalyst has been produced by leaching and doping, the subsequently doped metal element is preferably on the outer active catalyst layers, which can likewise be abraded by mechanical stress caused by liquid or gas. If the promoter element is abraded, this can reduce activity and selectivity of the catalyst for consequence. It has now been found that, surprisingly, the original activity can be restored by conducting the doping operation again. Alternatively, the doping can also be added to the hydrogenation, in which case redoping is effected in situ (method 4).

The examples which follow serve to illustrate the invention, but without restricting it in any way. Unless stated otherwise, the reactors were operated in liquid phase mode.

### EXAMPLES

### Example 1

### Hydrogenation of 4-isobutylacetophenone to 1-(4′-isobutylphenyl)ethanol

A wire mesh in the form of a plain weave, composed of an aluminum-containing ferritic chromium steel alloyed with yttrium and hafnium and having materials no. 1.4767, with a mesh size of 0.18 mm and a wire diameter of 0.112 mm, was heat-treated under air at 1000° C. for 5 h. Subsequently, the support mesh thus pretreated was coated in an electron beam deposition system with 110 mg of copper/m² (based on the mesh area). The coated mesh was heated under air at 400° C. for 0.5 hour in a muffle furnace for formation of the catalyst. The catalyst mesh thus produced was used to form a monolithic body. For this purpose, by means of a toothed roller, a portion of the mesh was corrugated. This corrugated mesh was combined with a smooth fabric strip and coiled. This gave a monolithic shaped body which was fixed by point welding. The diameter of the coil was 2.5 cm, the length 20 cm. In the apparatus described hereinafter which was used for the hydrogenations as well, this catalyst was reduced with hydrogen at 180° C. at ambient pressure for one hour.

The hydrogenation apparatus consisted of a reservoir vessel, a feed pump, a compressed gas supply, a jacketed tubular reactor with an oil-heated jacket, a gas/liquid separator and a circulation pump. In the separator, the reactor output was separated into reactor offgas and liquid output, and the gas was discharged via a pressure-retaining valve and the liquid under level control (i.e. depending on the liquid level in the separator). The gas and reactant feed point was between the circulation pump and reactor inlet.

After the catalyst had been activated, the system was filled with ethanol and a circulation stream of 46 liters/h was pumped through the reactor. Subsequently, by means of gas supply, hydrogen with a CO content of two ppm by volume was fed in and the reactor was brought to pressure 50 bar and heated up to 120° C. For the attainment of this temperature, a feed of 4-isobutylacetophenone of 31 g/h was established and about 4.7 standard liters of gas/h were fed in. The offgas rate was about 0.6 standard liter/h with a CO content of about 10 ppm by volume. After an operating time of 48 h, analysis of the output showed a conversion of 4-isobutylacetophenone of 99.7% with a selectivity of >99.5%. A secondary component obtained was 4-isobutylethylbenzene. The hydrogenation was operated for 10 days without occurrence of any impairment of catalyst activity and selectivity.

### Comparative Example 1

The procedure was analogous to example 1, except that the CO content was less than 0.1 ppm by volume at the reactor inlet and at the reactor outlet. The conversion of 4-isobutylacetophenone was 99.9% with a selectivity of about 97%. A secondary component with a selectivity of about 2.5% was 4-isobutylethylbenzene, and about 0.4% 1-(4′-isobutylcyclohexyl)ethanol.

### Example 2

### Hydrogenation of butyne-1,4-diol

The shaped nickel-aluminum catalyst bodies used in the application examples were prepared on the basis of the examples for preparation of catalyst foams present in EP 2 764 916 A1:

Variant a):

0.5 g of polyvinylpyrrolidone (molar mass: 40 000 g/mol) were dissolved in 29.5 g of demineralized water, and 20 g of aluminum powder (particle size 75 μm) were added. The mixture obtained was subsequently agitated, so as to give a homogeneous suspension. Thereafter, a nickel foam having an average pore size of 580 μm, a thickness of 1.9 mm and a basis weight of 1000 g/m² was introduced into the suspension, which was agitated vigorously again. The foam thus coated was placed onto a paper towel and the excess suspension was cautiously dabbed off. In a rotary kiln, the foam thus coated was heated up to 300° C. at a heating rate of 5° C./min, then kept at 300° C. under isothermal conditions for 30 min, heated further to 600° C. at 5° C./min, kept under isothermal conditions for 30 min and heated further to 700° C. at 5° C./min and kept under isothermal conditions for 30 min. The heating was effected in a gas stream that consisted of 20 L (STP)/h of nitrogen and 20 L (STP)/h of hydrogen. The cooling phase down to a temperature of 200° C. was likewise effected in a gas stream composed of 20 L (STP)/h of N₂ and 20 L (STP)/h of H₂. Thereafter, further cooling was effected to room temperature in a stream of 100 L (STP)/h of nitrogen. The foam thus produced had a decrease in weight of 42% compared to the nickel foam originally used.

Variant b):

A nickel foam having an average pore size of 580 μm, a thickness of 1.9 mm and a basis weight of 1000 g/m² was immersed into a 1% by weight polyvinylpyrrolidone solution (molar mass: 40 000 g/mol). After the immersion, the foam was squeezed on a flow cloth in order to remove the binder from the cavities of the pores. The foam laden with the binder was then clamped in an agitator and coated with aluminum powder (particle size <75 μm). The agitation resulted in a homogeneous distribution of the powder on the surface of the open-pore foam structure, followed by removal of excess aluminum powder. In a rotary kiln, the foam thus coated was heated up to 300° C. at a heating rate of 5° C./min, then kept at 300° C. under isothermal conditions for 30 min, heated further to 600° C. at 5° C./min, kept under isothermal conditions for 30 min and heated further to 700° C. at 5° C./min and kept under isothermal conditions for 30 min. The heating was effected in a gas stream that consisted of 20 L (STP)/h of nitrogen and 20 L (STP)/h of hydrogen. The cooling phase down to a temperature of 200° C. was likewise effected in a gas stream composed of 20 L (STP)/h of N₂ and 20 L (STP)/h of H₂. Thereafter, further cooling was effected down to room temperature in a stream of 100 L (STP)/h of nitrogen. The foam thus produced had a decrease in weight of 36% compared to the nickel foam originally used.

30 (about 57 mL) of the cuboidal shaped bodies obtained according to variant a) were installed into a rectangular reactor (interior dimensions 1 cm×2 cm×40 cm), such that 5 cuboids each lay flat one on top of another and 8 of these layers were stacked one on top of another in the reactor. In order that the stacks did not have any empty space with respect to the reactor wall, the interspace was filled and sealed with PTFE tape (thickness=0.5 cm). Thus, at least 95% of the reactor cross section was filled by the fixed catalyst bed comprising the shaped catalyst bodies, and 98% of the pores and channels had an area of not more than 0.7 mm². The reactor used also had an oil-heatable jacket. In addition, the experimental apparatus also comprised a level-regulated gas/liquid separator, a circulation pump, a feed pump for aqueous butyne-1,4-diol, a hydrogen supply, pressure valves in the offgas and liquid output, and devices for temperature measurement and sampling points.

The reactor and the circulation flow were filled with demineralized water and a circulation flow rate of about 20 L/h was established at standard pressure and 25° C. Thereafter, about 650 mL/h of a 0.5% by weight sodium hydroxide solution were fed in. Hydrogen released and excess liquid were discharged from the separator, and the residual liquid was recycled into the reactor. The nickel content in the liquid discharge stream was below the detection limit of 1 ppm. After about 6 h, the evolution of hydrogen noticeably declined and the feed of sodium hydroxide solution was stopped and then purging was effected with 5 L/h of demineralized water until the pH in the output was 7 and the conductivity had dropped to 254 pS/cm.

Subsequently, 0.6 g of (NH₄)₆Mo₇O₂₄×4 H₂O in 60 mL of water was fed in in liquid phase mode via the circulation pump, again at 25° C. The solution was pumped in circulation at 20 kg/h for 3 h until the Mo had been absorbed by the catalyst.

For hydrogenation, a butyne-1,4-diol starting material which had been prepared according to example 1 of EP 2121549 A1 was used. The starting material had a pH of 7.5 and comprised, as well as butyne-1 ,4-diol and water, also about 1% by weight of propynol, 1.2% by weight of formaldehyde and a number of other by-products having proportions of well below 1% by weight. The hydrogenation was conducted at pressure 45 bar and a temperature of 155 to 160° C. and a circulation flow rate of 13 to 20 kg/h. The molar ratio of hydrogen to butyne-1,4-diol was 2.5:1. Table 1 shows the results (each after a run time of 48 h with appropriate adjustment of experimental parameters), the GC percentages being area % (i.e. water was not taken into account). The CO concentration is reported in ppm by volume at the start and at the end of the reactor.

The CO concentration is reported in ppm by volume at the start and at the end of the reactor.

### Comparative Example 2

Cuboidal shape catalyst bodies according to variant a) were produced as described in example 2. 30 cuboids were cut into pieces of approximate size 2×2×1.9 mm and introduced as a bed (about 70 mL) into the reactor likewise described in example 2 and activated and doped with Mo analogously to example 2. The reactor cross section was about 80% filled with shaped catalyst bodies and about 10% of the channels had at least an area of 0.7 mm². The hydrogenation of butyne-1,4-diol was conducted at a space velocity of 0.5 kg of butyne-1,4-diol/L×h and a circulation flow rate of 20 kg/h analogously to example 2. The hydrogenation proceeded, in terms of selectivity and conversion, much less efficiently than in the inventive example 2 (see table 2).

TABLE 1
(according to the invention)

| Catalyst hourly | Circulation | | | | | | | | | |
|---|---|---|---|---|---|---|---|---|---|---|
| space velocity | flow rate | GC analysis (area %, no water) | | | | | | | Water (% | CO (ppm |
| (kg_{BYD}/(L_{cat}*h)) | (kg/h) | BDO | BuOH | MeOH | PrOH | MBDO | BED | BYD | by wt.) | by vol.) |
| 0.5 | 20 | 94.6 | 1.6 | 1.2 | 1.2 | 0.12 | 0 | 0 | 50 | 0.2-25 |
| 1.0 | 20 | 94.0 | 2.2 | 1.2 | 0.9 | 0.11 | 0 | 0 | 50 | 0.2-40 |
| 0.5 | 18 | 92.0 | 2.7 | 2.1 | 1.9 | 0.22 | 0 | 0 | 50 | 0.2-50 |
| 0.5 | 13 | 91.9 | 2.9 | 1.3 | 1.8 | 0.25 | 0 | 0 | 50 | 0.2-70 |

TABLE 2
(not according to the invention)

| Catalyst hourly | Circulation | | | | | | | | | |
|---|---|---|---|---|---|---|---|---|---|---|
| space velocity | flow rate | GC analysis (area %, no water) | | | | | | | Water (% | CO (ppm |
| (kg_{BYD}/(L_{cat}*h)) | (kg/h) | BDO | BuOH | MeOH | PrOH | MBDO | BED | BYD | by wt.) | by vol.) |
| 0.5 | 20 | 84.3 | 5.9 | 0.7 | 0.2 | 0.3 | 4.2 | 0 | 50 | 50-7500 |

BDO = butane-1,4-diol
BuOH = n-butanol
MeOH = methanol
PrOH = n-propanol
MBDO = 2-methylbutane-1,4-diol
BYD = but-2-yne-1,4-diol
BED = but-2-ene-1,4-diol

### Example 3

### Hydrogenation of n-butyraldehyde

Analogously to example 2, n-butyraldehyde that still comprised about 1500 ppm of isobutyraldehyde was hydrogenated over nickel-aluminum cuboids (produced according to variant b)), the activation being followed by doping with Mo in reverse flow direction through the reactor relative to that in example 2. Thereafter, the reaction system was emptied and the water was replaced by n-butanol. The hydrogenation was effected at 40 bar, in the course of which the temperature was kept between 135 and 140° C. The molar ratio of hydrogen to butyraldehyde was 1.1:1. At a space velocity of 1.5 kg of butyraldehyde/liter of catalyst x h and a liquid circulation rate of 23 kg/h, according to GC analysis (area%), 99.6% n-butanol was found in the output. In addition, main secondary components present were 0.05% butyl acetate, 0.01% dibutyl ether, 0.15% isobutanol and 0.07% ethylhexanediol. The CO content upstream of the reactor was 0.2 ppm by volume, and downstream of the reactor 15 ppm by volume.

1. A process for hydrogenating a hydrogenatable organic compound in at least one reactor comprising a fixed catalyst bed comprising monolithic shaped catalyst bodies or consisting of monolithic shaped catalyst bodies comprising at least one element selected from Ni, Fe, Co, Cu, Cr, Pt, Ag, Au, Pd, Mn, Re, Ru, Rh and Ir, wherein the CO content in the gas phase within the reactor during the hydrogenation is within a range from 0.1 to 10 000 ppm by volume and wherein the fixed catalyst bed comprises shaped catalyst bodies having pores and/or channels, and wherein, in any section in the normal plane to flow direction through the fixed catalyst bed, at least 90% of the pores and channels, have an area of not more than 3 mm².

2. The process according to claim 1, wherein the compound used for hydrogenation is selected from butyne-1,4-diol, butene-1,4-diol, 4-hydroxybutyraldehyde, hydroxypivalic acid, hydroxypivalaldehyde, n-butyraldehyde, isobutyraldehyde, n-valeraldehyde, isovaleraldehyde, 2-ethylhex-2-enal, 2-ethylhexanal, the isomeric nonanals, cyclododeca-1,5,9-triene, benzene, furan, furfural, phthalic esters, acetophenone and alkyl-substituted acetophenones.

3. The process according to claim 1, wherein the conversion in the hydrogenation is at least 90 mol % based on the total molar amount of hydrogenatable components in the starting material used for hydrogenation.

4. The process according to claim 1, wherein, during the hydrogenation, the CO content in the gas phase within the reactor is within a range from 0.15 to 5000 ppm by volume.

5. The process according to claim 1, wherein the reactor has a gradient with respect to the CO concentration in flow direction of the reaction medium through the fixed catalyst bed.

6. The process according to claim 1, wherein the CO content on exit of the reaction medium from the fixed catalyst bed is at least 5 mol % higher than the CO content on entry of the reaction medium into the catalytically active fixed bed.

7. The process according to claim 1, wherein the fixed catalyst bed has, in any section in the normal plane to flow direction through the fixed catalyst bed, based on the total area of the section, not more than 5% free area that is not part of the shaped catalyst bodies.

8. The process according to claim 1, wherein the flow rate of the liquid reaction mixture through the reactor comprising the fixed catalyst bed is at least 30 m/h.

9. The process according to claim 1, wherein the reaction mixture for the hydrogenation is at least partly conducted in a liquid circulation stream, wherein the ratio of reaction mixture conducted in the circulation stream to freshly supplied reactant stream is within a range from 1:1 to 1000:1.

10. The process according to claim 1, wherein the fixed catalyst bed has a temperature gradient during the hydrogenation.

11. The process according to claim 1, wherein the monolithic shaped catalyst bodies, based on the overall shaped body, have a greatest dimension in any direction of at least 1 cm.

12. The process according to claim 1, wherein the monolithic shaped catalyst bodies comprise at least one element selected from Ni, Fe, Co, Cu, Cr, Pt, Ag, and Au.

13. The process according to claim 1, wherein the monolithic shaped catalyst bodies are in the form of a foam.

14. The process according to claim 1, wherein the reactor used for hydrogenation comprises a fixed catalyst bed comprising monolithic shaped catalyst bodies or consisting of monolithic shaped catalyst bodies comprising at least one first metal selected from Ni, Fe, Co, Cu, Cr, Pt, Ag, and Au, and comprising at least one second component selected from Al, Zn and Si, wherein the fixed catalyst bed is activated by subjecting it to a treatment with an aqueous base, in which
a) a fixed catalyst bed comprising monolithic shaped catalyst bodies or consisting of monolithic shaped catalyst bodies comprising at least one first metal selected from Ni, Fe, Co, Cu, Cr, Pt, Ag, and Au, and comprising at least one second component selected from Al, Zn and Si, is introduced into a reactor,
b) the fixed catalyst bed, for activation, is subjected to a treatment with an aqueous base,
c) the activated fixed catalyst bed obtained in step b) is optionally subjected to a treatment with a wash medium selected from water, C1-C4-alkanols and mixtures thereof,
d) the fixed catalyst bed obtained after the activation in step b) or after the treatment in step c) is optionally contacted with a dopant including at least one element other than the first metal and the second component of the shaped catalyst bodies used in step a).

15. The process according to claim 1, wherein in any section in the normal plane to flow direction through the fixed catalyst bed, at least 98% of the pores and channels have an area of not more than 3 mm².

16. The process according to claim 3, wherein the conversion in the hydrogenation is at least 95 mol % based on the total molar amount of hydrogenatable components in the starting material used for hydrogenation.

17. The process according to claim 4, wherein, during the hydrogenation, the CO content in the gas phase within the reactor is within a range 0.2 to 1000 ppm by volume.

18. The process according to claim 6, wherein the CO content on exit of the reaction medium from the fixed catalyst bed is at least 25 mol % higher than the CO content on entry of the reaction medium into the catalytically active fixed bed.

19. The process according to claim 7, wherein the fixed catalyst bed has, in any section in the normal plane to flow direction through the fixed catalyst bed, based on the total area of the section, not more than 1% free area that is not part of the shaped catalyst bodies.

20. The process according to claim 8, wherein the flow rate of the liquid reaction mixture through the reactor comprising the fixed catalyst bed is at least 50 m/h.
